(19) United States
(12) Patent Application Publication
(10) Pub. No.: US 2016/0250177 A1
(43) Pub. Date: Sep. 1, 2016

(54) MODIFIED DOCETAXEL LIPOSOME FORMULATIONS AND USES THEREOF

(71) Applicant: MALLINCKRODT LLC, HAZELWOOD, MO (US)

(72) Inventors: WILLIAM McGHEE, FENTON, MO (US); JAMES BLACKLEDGE, ST. LOUIS, MO (US); MARGARET GRAPPERHAUS, TROY, IL (US); LOUISE ROCHON, ST. LOUIS, MO (US); KRISHNA DEVARAKONDA, ST. LOUIS, MO (US)

(21) Appl. No.: 15/046,243

(22) Filed: Feb. 17, 2016

Related U.S. Application Data
(60) Provisional application No. 62/117,299, filed on Feb. 17, 2015.
(60) Provisional application No. 62/148,549, filed on Apr. 16, 2015.

(51) Int. Cl. A61K 31/337; A61K 47/24; A61K 47/12; A61K 9/127; A61K 47/28

(52) U.S. Cl. CPC A61K 31/337; A61K 9/127; A61K 47/28; A61K 47/12; A61K 47/24

(57) ABSTRACT

The present invention provides compositions for the treatment of cancer. The compositions include liposomes containing a phosphatidylcholine lipid, a sterol, a PEG-lipid, and a taxane. The PEG-lipid constitutes from about 2 to about 8 mol % of the lipids in the liposome. The taxane is docetaxel esterified at the 2′-O position with a heterocyclyl-(C₂₋₅ alkanoic acid). The present invention also provides liposomal compositions for the treatment of cancer comprising administering to a patient in need thereof a liposome, wherein the liposome comprises: a phosphatidylcholine lipid; a sterol; a PEG-lipid; and a taxane or a pharmaceutically acceptable salt thereof; wherein the taxane is docetaxel esterified at the 2′-O-position with a heterocyclyl-(C₂₋₅alkanoic acid); and wherein upon administration of the liposomal composition to the patient, the plasma concentration of docetaxel remain above an efficacy threshold of 0.2 μM for at least 5 hours.

### CROSS-REFERENCE TO RELATED APPLICATIONS

This application claims priority to U.S. Provisional Application No. 62/117,299 filed on Feb. 17, 2015 and U.S. Provisional Application No. 62/148,549 filed on Apr. 16, 2015, which are incorporated herein by reference in their entirety to the full extent permitted by law.

### BACKGROUND OF THE INVENTION

Taxotere® (docetaxel) and Taxol® (paclitaxel) are the most widely prescribed anticancer drugs on the market, and are associated with a number of pharmacological and toxicological concerns, including highly variable (docetaxel) and non-linear (paclitaxel) pharmacokinetics (PK), serious hypersensitivity reactions associated with the formulation vehicle (Cremophor EL, Tween 80), acute and dose-limited toxicities, such as myelosuppression, neurotoxicity, fluid retention, asthenia, hyperlacrimation, oncholysis and alopecia. In the case of Taxotere®, the large variability in PK causes significant variability in toxicity and efficacy, as well as hematological toxicity correlated with systemic exposure to the unbound drug. In addition, since the therapeutic activity of taxanes increases with the duration of tumor cell drug exposure, the dose-limiting toxicity of commercial taxane formulations substantially limits their therapeutic potential. Resistance to the drugs due to causes, such as up-regulation of protein transporter pumps by cancer cells, can further complicate taxane-based therapies. As such, there exists a need for taxane-based chemotherapeutics with decreased toxicity and improved efficacy. The present invention addresses this and other needs.

### BRIEF SUMMARY OF THE INVENTION

In one aspect, the present invention provides a composition for the treatment of cancer. The composition includes a liposome containing a phosphatidylcholine lipid, a sterol, a poly(ethylene glycol)-phospholipid conjugate (PEG-lipid) and a taxane or a pharmaceutically acceptable salt thereof. The taxane is docetaxel esterified at the 2′-O-position with a heterocyclyl-(C₂₋₅ alkanoic acid), and the PEG-lipid constitutes 2-8 mol % of the total lipids in the liposome.

In another aspect, the invention provides a method for preparing a liposomal taxane. The method includes: a) forming a first liposome having a lipid bilayer including a phosphatidylcholine lipid and a sterol, wherein the lipid bilayer encapsulates an interior compartment comprising an aqueous solution; b) loading the first liposome with a taxane, or a pharmaceutically acceptable salt thereof, to form a loaded liposome, wherein the taxane is docetaxel esterified at the 2′-O-position with a heterocyclyl-(C₂₋₅ alkanoic acid); and c) forming a mixture containing the loaded liposome and a PEG-lipid under conditions sufficient to allow insertion of the PEG-lipid into the lipid bilayer.

In still another aspect, the invention provides liposomal compositions for the treatment of cancer comprising administering to a patient in need thereof a liposome, wherein the liposome comprises: a phosphatidylcholine lipid; a sterol; a PEG-lipid; and a taxane or a pharmaceutically acceptable salt thereof; wherein the taxane is docetaxel esterified at the 2′-O-position with a heterocyclyl-(C₂₋₅ alkanoic acid); and wherein upon administration of the liposomal composition to the patient, the plasma concentration of docetaxel remain above an efficacy threshold of 0.2 μM for at least 5 hours.

In yet another aspect, the invention provides a method for treating cancer. The method includes administering to a patient in need thereof the liposomal taxane composition of the present invention. In one embodiment, the liposome comprises: a phosphatidylcholine lipid; a sterol; a PEG-lipid; and a taxane or a pharmaceutically acceptable salt thereof; wherein the taxane is docetaxel esterified at the 2′-O-position with a heterocyclyl-(C₂₋₅ alkanoic acid); and wherein upon administration of the liposomal composition to the patient, the plasma concentration of docetaxel remains above an efficacy threshold of 0.2 μM for at least 5 hours.

### REFERENCE TO COLOR FIGURES

This application file contains at least one drawing executed in color. Copies of this patent application publication with color drawings will be provided by the Office upon request and payment of the necessary fee.

### BRIEF DESCRIPTION OF THE DRAWINGS

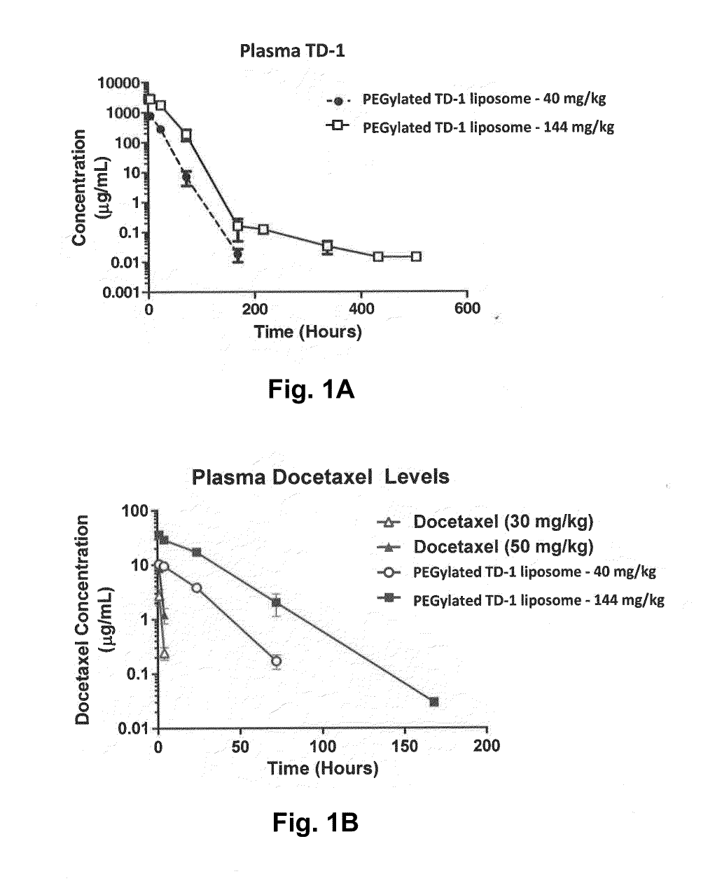

FIG. 1A shows the clearance of TD-1 from plasma following administration of PEGylated TD-1 liposomes to mice bearing A549 xenograft. FIG. 1B shows the clearance of docetaxel from plasma following administration of PEGylated TD-1 liposomes to mice bearing A549 xenograft. Data are represented as mean±standard error of three mice or as the mean or single value if less than three mice.

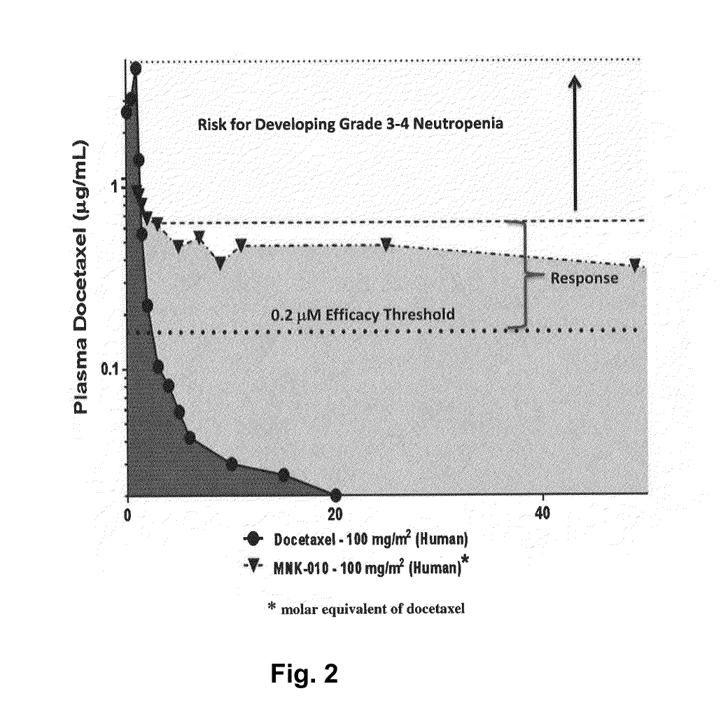

FIG. 2 shows the plasma concentration of docetaxel following administration of the molar equivalent of docetaxel released from PEGylated TD-1 liposomes (100 mg/m²) and docetaxel (100 mg/m²). Data are represented a single value.

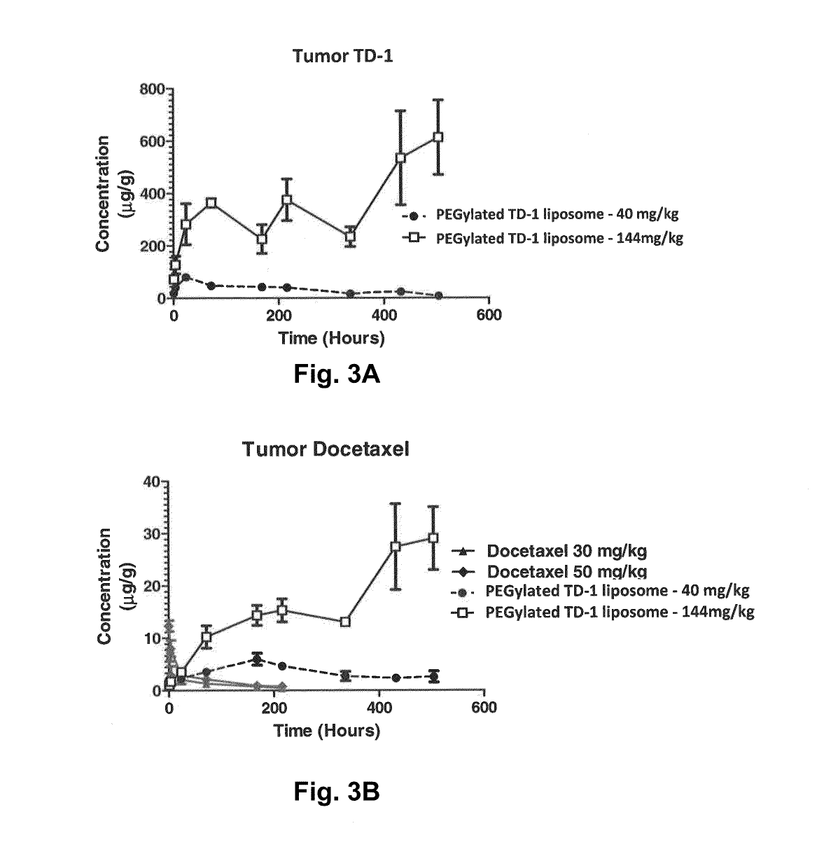

FIG. 3A shows the levels of TD-1 in tumors following administration of PEGylated TD-1 liposomes to mice bearing A549 human NSCLC xenograft. FIG. 3B shows the levels of PEGylated TD-1 liposomes and docetaxel in tumors following administration of PEGylated TD-1 liposomes and docetaxel to mice bearing A549 human NSCLC xenograft. Data are represented as mean±standard error of three mice or as the mean or single value if less than three mice.

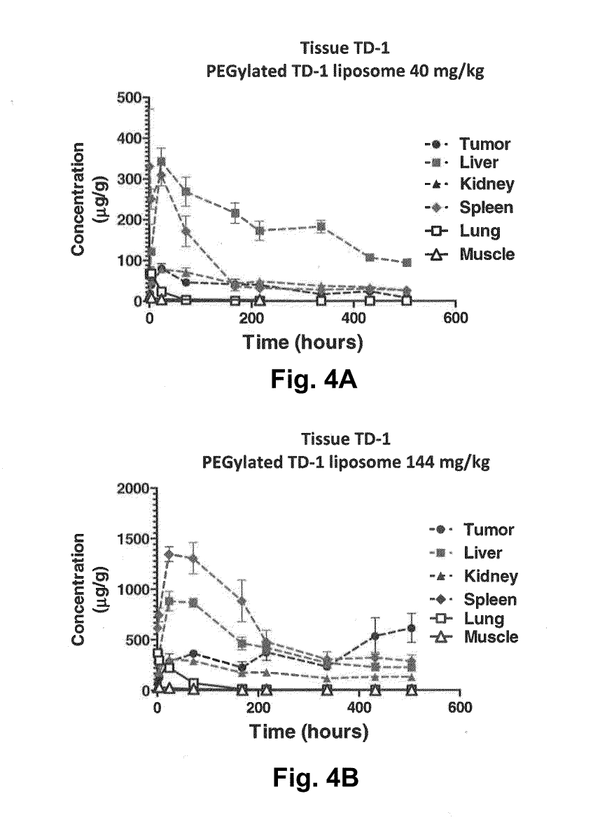

FIG. 4A shows the levels of TD-1 over time in tissue following administration of 40 mg/kg PEGylated TD-1 liposomes to mice bearing A549 human NSCLC xenograft. FIG. 4B shows the levels of TD-1 over time in tissue following administration of 144 mg/kg PEGylated TD-1 liposomes to mice bearing A549 human NSCLC xenograft. Data are represented as mean±standard error of three mice or as the mean or single value if less than three mice.

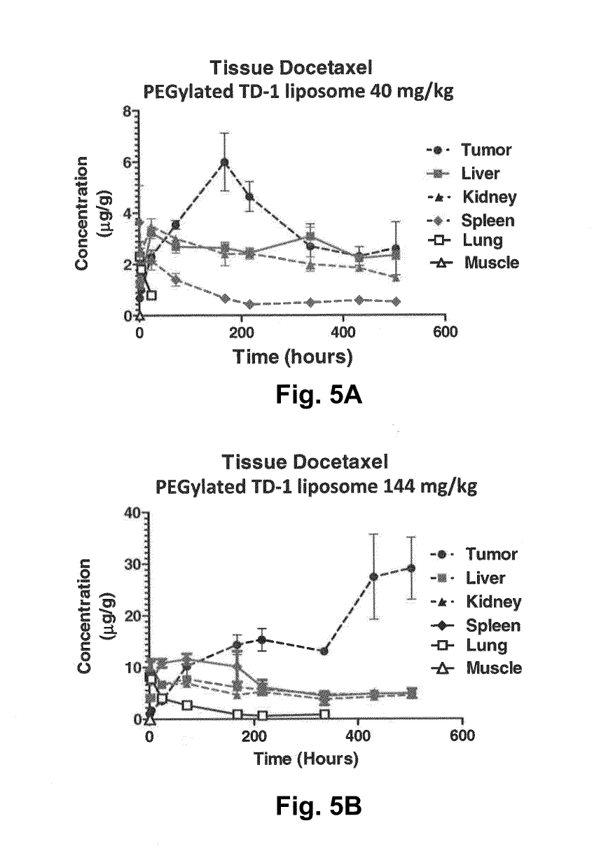

FIG. 5A shows the levels of docetaxel over time in tissue following administration of 40 mg/kg PEGylated TD-1 liposomes to mice bearing A549 human NSCLC xenograft. FIG. 5B shows the levels of docetaxel over time in tissue following administration of 144 mg/kg PEGylated TD-1 liposomes to mice bearing A549 human NSCLC xenograft. Data are represented as mean±standard error of three mice or as the mean or single value if less than three mice.

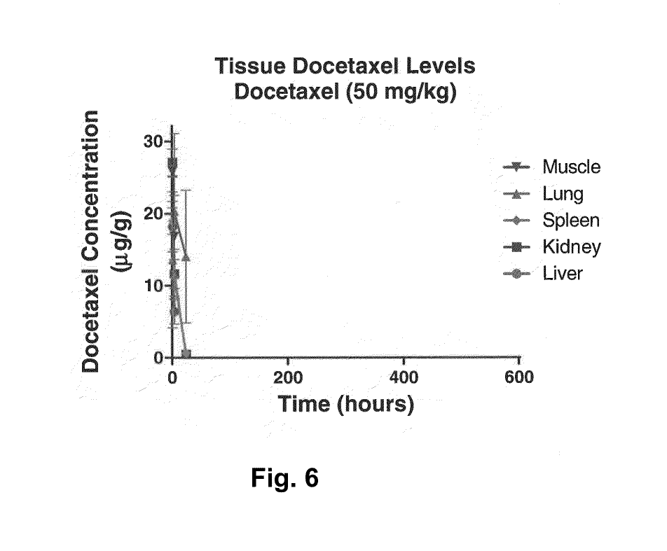

FIG. 6 shows the levels of docetaxel over time in tissue following administration of 50 mg/kg docetaxel to mice bearing A549 human NSCLC xenograft. Data are represented as mean±standard error of three mice or as the mean or single value if less than three mice.

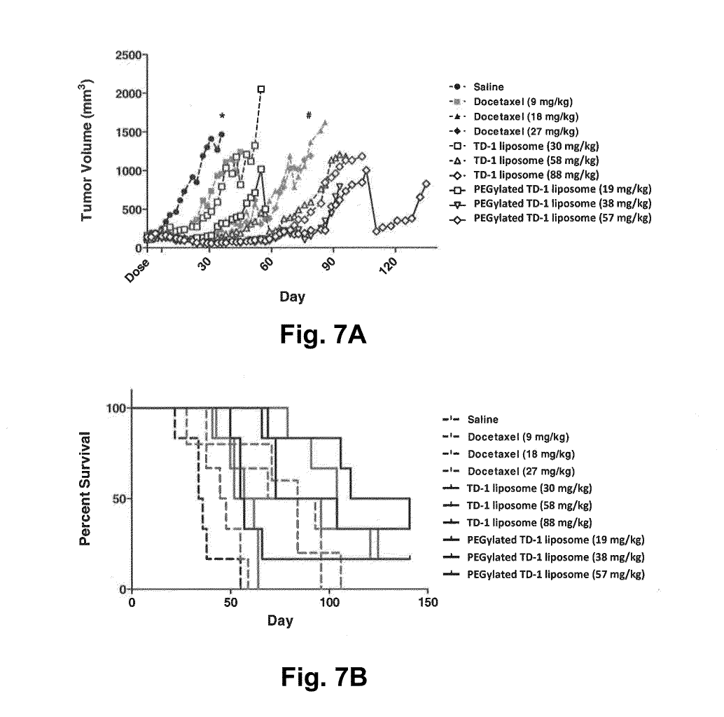

FIG. 7A shows the antitumor effect of TD-1 liposomes, PEGylated TD-1 liposomes and docetaxel against human PC3 (prostate) tumor xenograft in athymic nude mice. All treatment groups exhibited significantly smaller tumors than saline 36 days following a single IV administration. Treatment with PEGylated TD-1 liposomes at 19 mg/kg caused significantly smaller tumors than the equitoxic dose of docetaxel (9 mg/kg) and TD-1 liposomes (30 mg/kg), *, p<0.05. PEGylated TD-1 liposomes (38 mg/kg) caused smaller tumors than docetaxel (18 mg/kg) at comparably tolerated doses on day 79 post treatment, #, p<0.05. Analysis was conducted using one-way ANOVA followed by a Newman-Keuls post hoc test. Data are represented as mean of three to six mice.

FIG. 7B shows a Kaplan-Meier survival plot of athymic nude mice bearing human PC3 (prostate) xenograft tumors treated with TD-1 liposomes, PEGylated TD-1 liposomes, docetaxel or saline. Docetaxel treatment at 18 and 27 mg/kg and all treatment doses of TD-1 liposomes and PEGylated TD-1 liposomes increased survival significantly more than saline, p<0.05, Mantel-Cox, log-rank test. Each group started with five to six male mice bearing tumors.

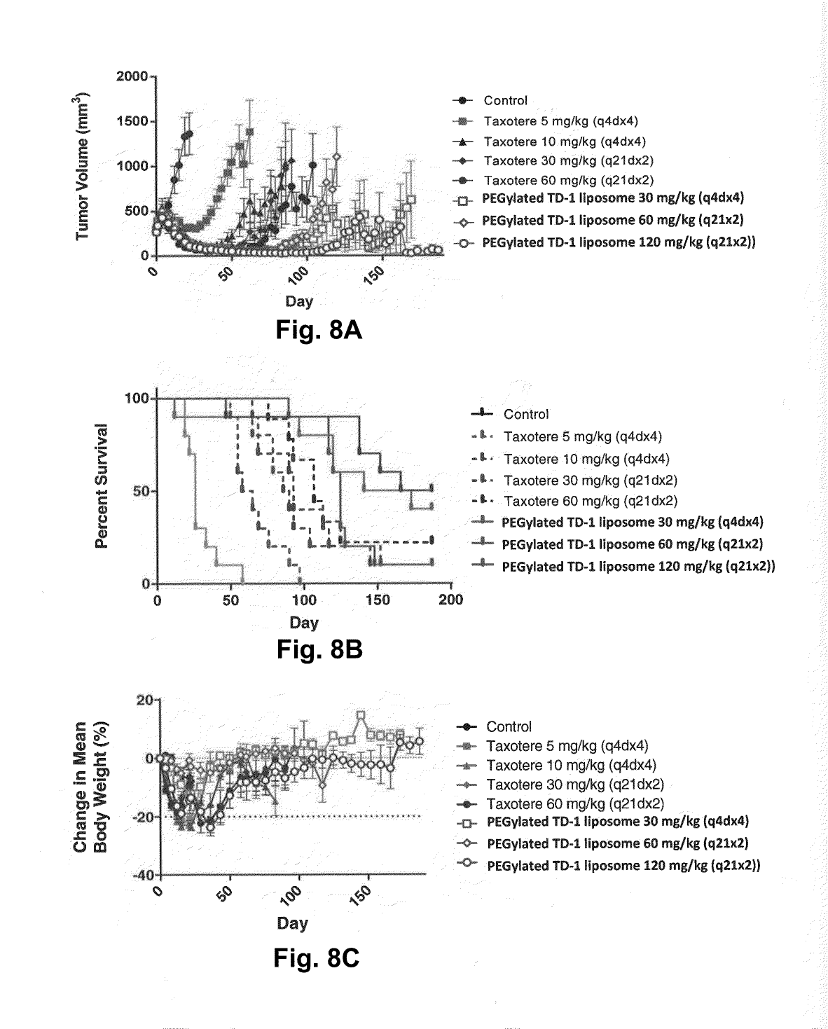

FIG. 8A shows the antitumor effect of PEGylated TD-1 liposomes and docetaxel against human PC3 (prostate) tumor xenograft in athymic nude mice. All dose groups of PEGylated TD-1 liposomes inhibited tumor growth longer than all dose groups of docetaxel. Data are represented as mean of five to ten mice.

FIG. 8B shows a Kaplan-Meier survival plot of athymic nude mice bearing human PC3 (prostate) xenograft tumors treated with PEGylated TD-1 liposomes or docetaxel. All dose groups of PEGylated TD-1 liposomes increased median survival of mice greater than docetaxel. Data are represented as mean of five to ten mice.

FIG. 8C shows the body weight changes of athymic nude mice bearing human PC3 prostate xenograft tumors treated with PEGylated TD-1 liposomes or docetaxel. Data are represented as mean of five to ten mice.

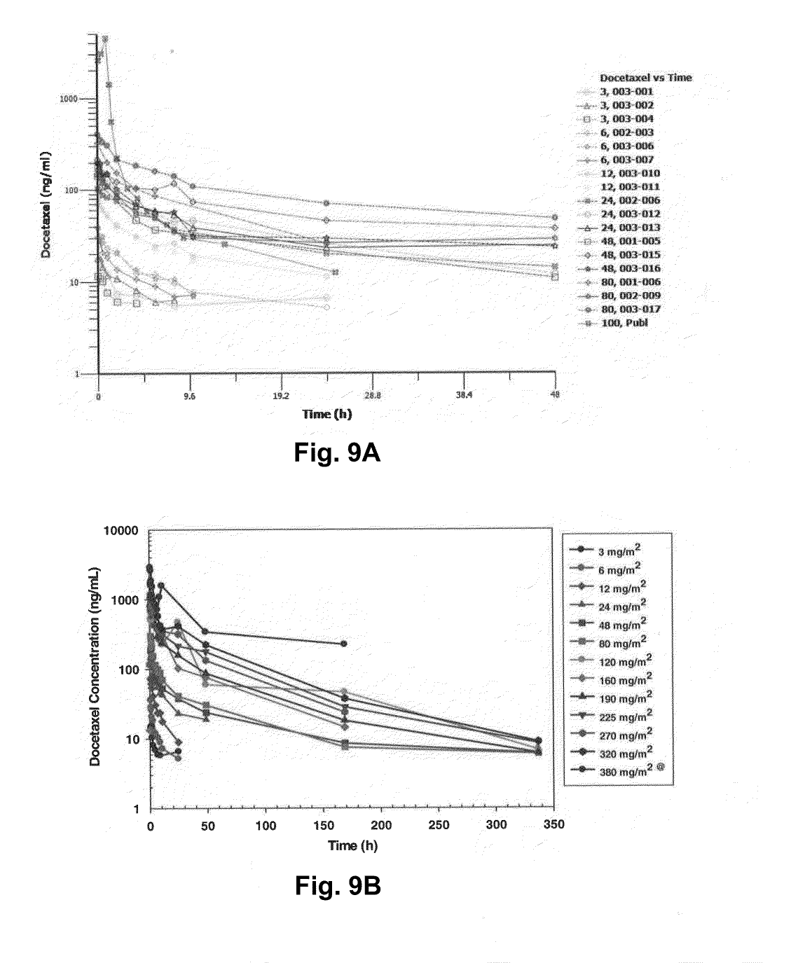

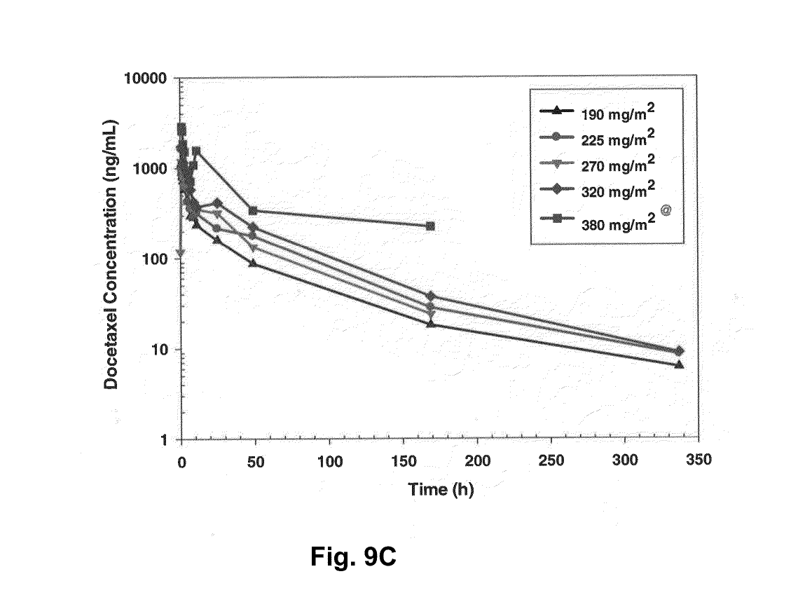

FIG. 9A shows the plasma concentration of docetaxel over time (48 hrs) following administration of PEGylated TD-1 liposomes at dose levels of 3, 6, 12, 24, 48, and 80 mg/m², and a published report of plasma concentration of docetaxel at a dose of 100 mg/m². Data are represented as single values.

FIG. 9B shows the plasma concentration of docetaxel over time following administration of PEGylated TD-1 liposomes at dose levels of 3, 6, 12, 24, 48, 80, 120, 160, 190, 225, 270, 320 and 380 mg/m². Data are represented as mean of three mice, except for 380 mg/m² which is a single value.

FIG. 9C shows the plasma concentration of docetaxel over time following administration of PEGylated TD-1 liposomes at dose levels of 190, 225, 270, 320 and 380 mg/m². Data are represented as mean of three mice, except for 380 mg/m² which is a single value.

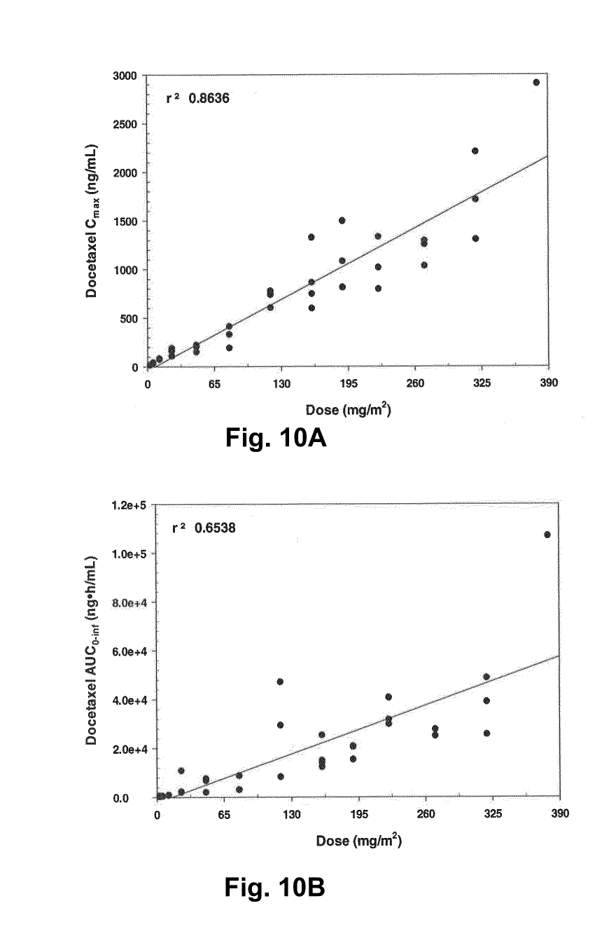

FIG. 10A shows the correlation between peak docetaxel concentration (Cₘₐₓ) and dose levels administered at 3, 6, 12, 24, 48, 80, 120, 160, 190, 225, 270, 320 and 380 mg/m². Data are represented as mean of three mice, except for 380 mg/m² which is a single value.

FIG. 10B shows the correlation between docetaxel exposure (AUC_{0-inf}) and dose levels administered at 3, 6, 12, 24, 48, 80, 120, 160, 190, 225, 270, 320 and 380 mg/m². Data are represented as mean of three mice, except for 380 mg/m² which is a single value.

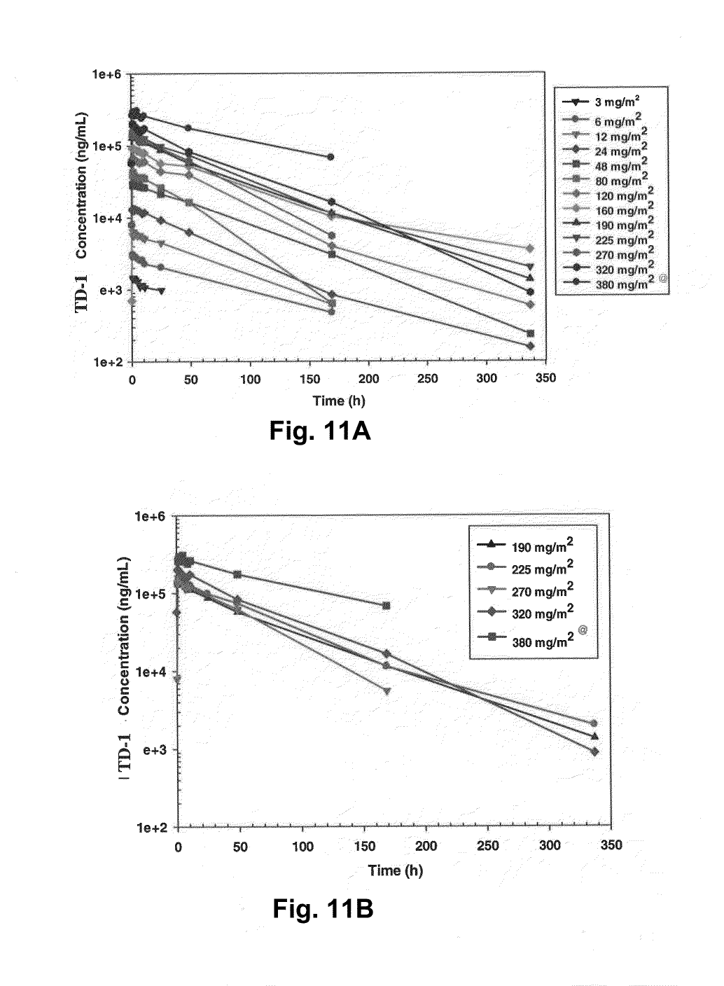

FIG. 11A shows the plasma concentration of TD-1 over time following administration of PEGylated TD-1 liposomes at dose levels of 3, 6, 12, 24, 48, 80, 120, 160, 190, 225, 270, 320 and 380 mg/m². Data are represented as mean of three mice, except for 380 mg/m² which is a single value.

FIG. 11B shows the plasma concentration of TD-1 over time following administration of PEGylated TD-1 liposomes at dose levels of 190, 225, 270, 320 and 380 mg/m². Data are represented as mean of three mice, except for 380 mg/m² which is a single value.

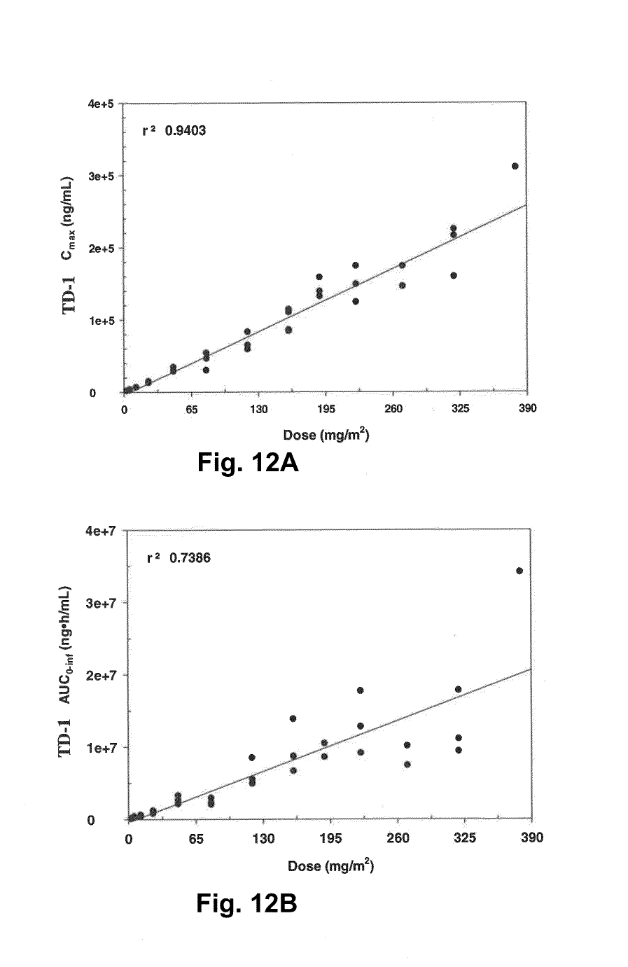

FIG. 12A shows the correlation between peak TD-1 concentration (Cₘₐₓ) and dose levels administered at 3, 6, 12, 24, 48, 80, 120, 160, 190, 225, 270, 320 and 380 mg/m². Data are represented as mean of three mice, except for 380 mg/m² which is a single value.

FIG. 12B shows the correlation between TD-1 exposure (AUC_{0-inf}) and dose levels administered at 3, 6, 12, 24, 48, 80, 120, 160, 190, 225, 270, 320 and 380 mg/m². Data are represented as mean of three mice, except for 380 mg/m² which is a single value.

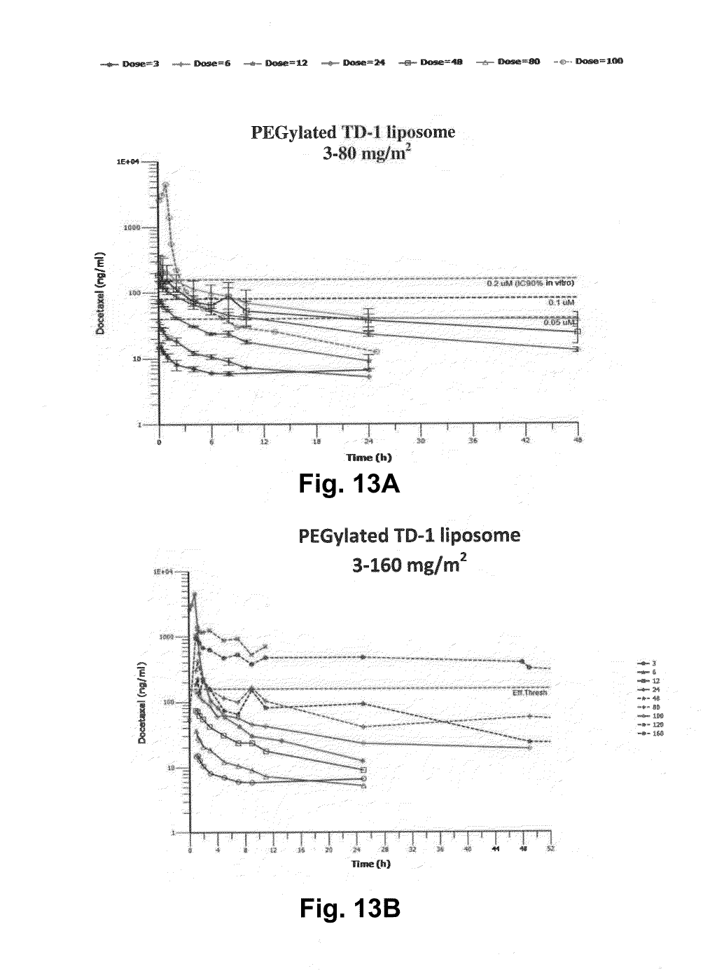

FIG. 13A shows the mean plasma concentration of docetaxel following administration of PEGylated TD-1 liposomes in cancer patients at dose levels of 3, 6, 12, 24, 48, & 80 mg/m². The putative efficacy threshold is provided. Data are represented as mean of two or three mice.

FIG. 13B shows the mean plasma concentration of docetaxel following administration of PEGylated TD-1 liposomes in cancer patients at dose levels of 3, 6, 12, 24, 48, 80, 120 & 160 mg/m². The putative efficacy threshold is provided. Data are represented as mean of two or three mice.

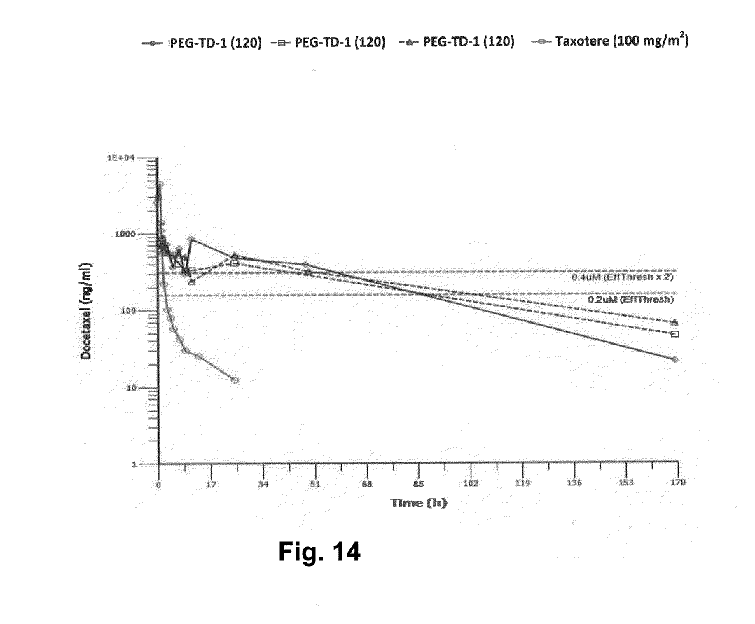

FIG. 14 shows the mean plasma concentration of docetaxel above the putative efficacy threshold (1× and 2×) following administration of PEGylated TD-1 liposomes (120 mg/m²) and Taxotere® (100 mg/m²) in cancer patients. Data are represented as single values.

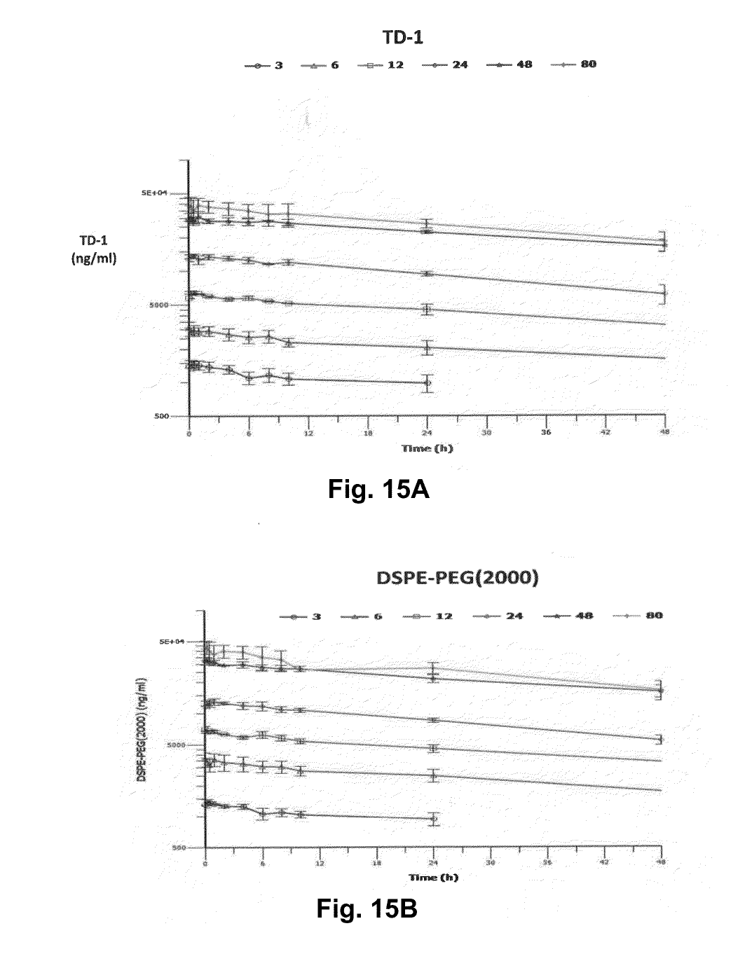

FIG. 15A shows the mean plasma concentration of TD-1 following administration of PEGylated TD-1 liposomes in cancer patients at dose levels of 3, 6, 12, 24, 48, & 80 mg/m². FIG. 15B shows the mean plasma concentration of DSPE-PEG(2000) following administration of PEGylated TD-1 liposomes in cancer patients at dose levels of 3, 6, 12, 24, 48, & 80 mg/m². Data are represented as mean of two or three mice.

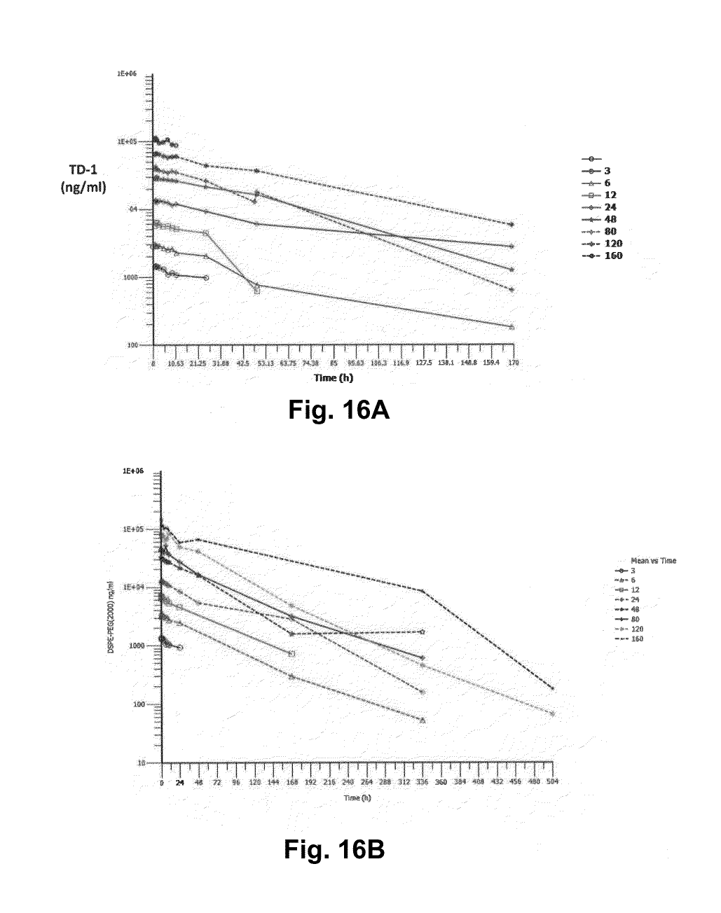

FIG. 16A shows the mean plasma concentration of TD-1 following administration of PEGylated TD-1 liposomes in cancer patients at dose levels of 3, 6, 12, 24, 48, 80, 120 & 160 mg/m². FIG. 16B shows the mean plasma concentration of DSPE-PEG(2000) following administration of PEGylated TD-1 liposomes in cancer patients at dose levels of 3, 6, 12, 24, 48, 80, 120 & 160 mg/m². Data are represented as mean of two or three mice.

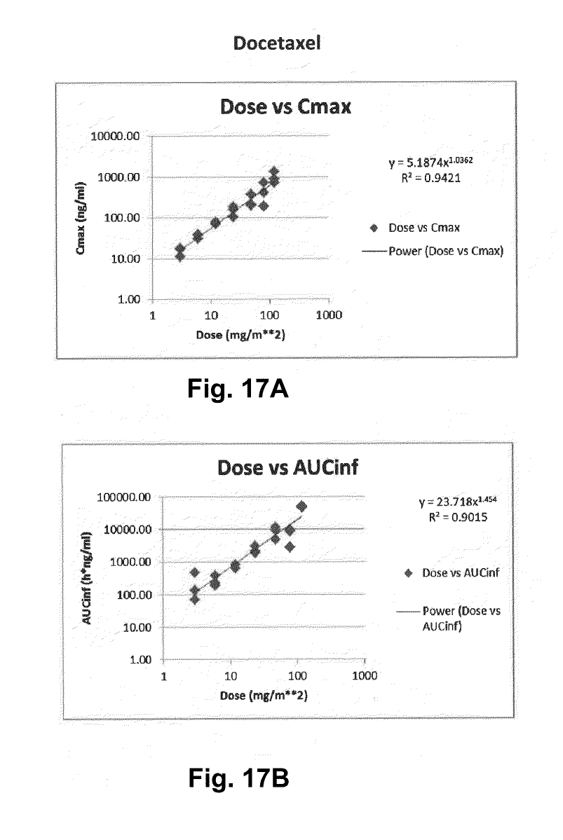

FIG. 17A shows pharmacokinetic dose proportionality of docetaxel following administration of PEGylated TD-1 liposomes in cancer patients for Cₘₐₓ. FIG. 17B shows pharmacokinetic dose proportionality of docetaxel following administration of PEGylated TD-1 liposomes in cancer patients for AUC_{inf} Data are represented as mean of two or three mice.

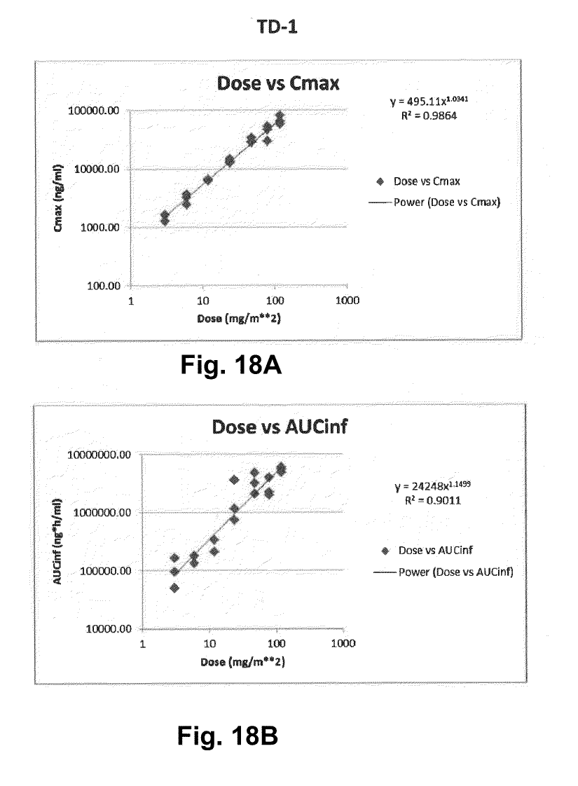

FIG. 18A shows pharmacokinetic dose proportionality for TD-1 following administration of PEGylated TD-1 liposomes in cancer patients for Cₘₐₓ. FIG. 18B shows pharmacokinetic dose proportionality for TD-1 following administration of PEGylated TD-1 liposomes in cancer patients for AUC_{inf}. Data are represented as mean of two or three mice.

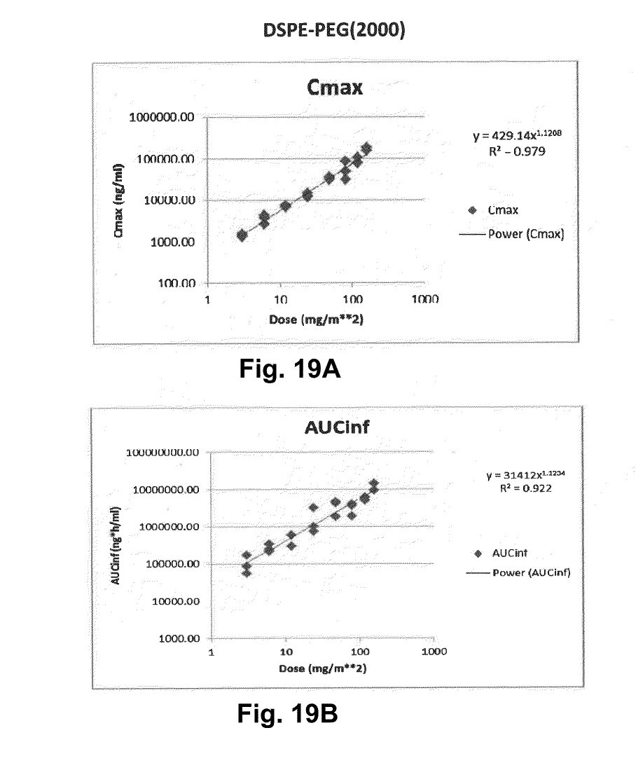

FIG. 19A shows pharmacokinetic dose proportionality for DSPE-PEG(2000) following administration of PEGylated TD-1 liposomes in cancer patients for Cₘₐₓ. FIG. 19B shows pharmacokinetic dose proportionality for DSPE-PEG(2000) following administration of PEGylated TD-1 liposomes in cancer patients for AUC_{inf}. Data are represented as mean of two or three mice.

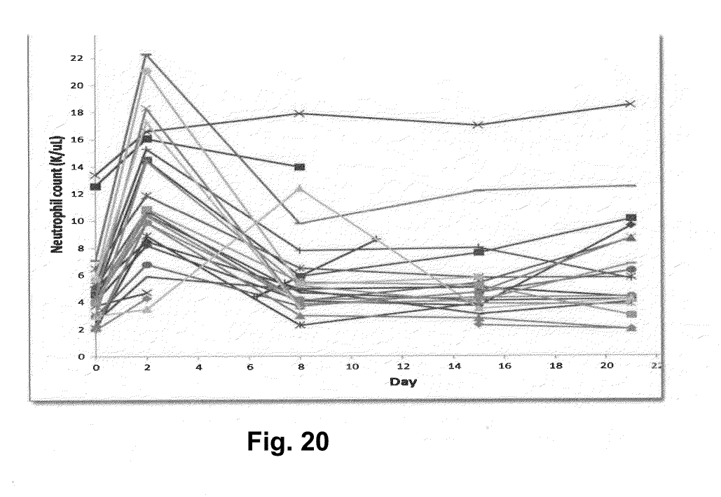

FIG. 20 shows the day vs. neutrophil count in patients treated with PEGylated TD-1 liposomes. Data are represented as single values.

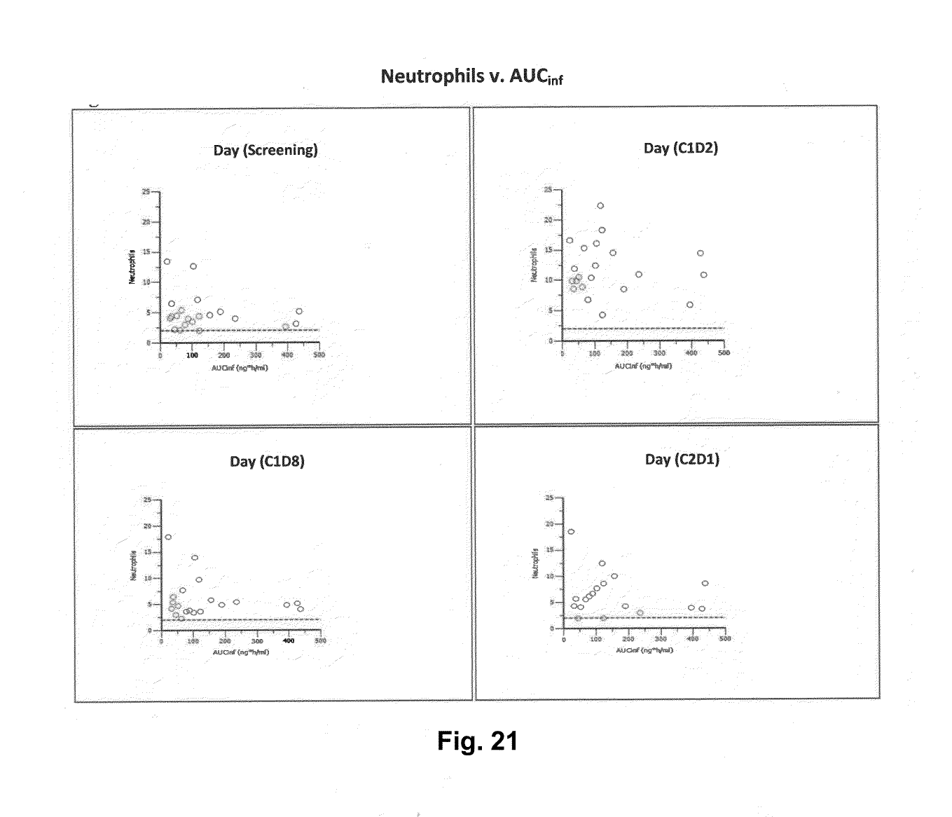

FIG. 21 shows the toxicity correlation between docetaxel AUC_{inf} and neutrophils in cancer patients. Data are represented as single values.

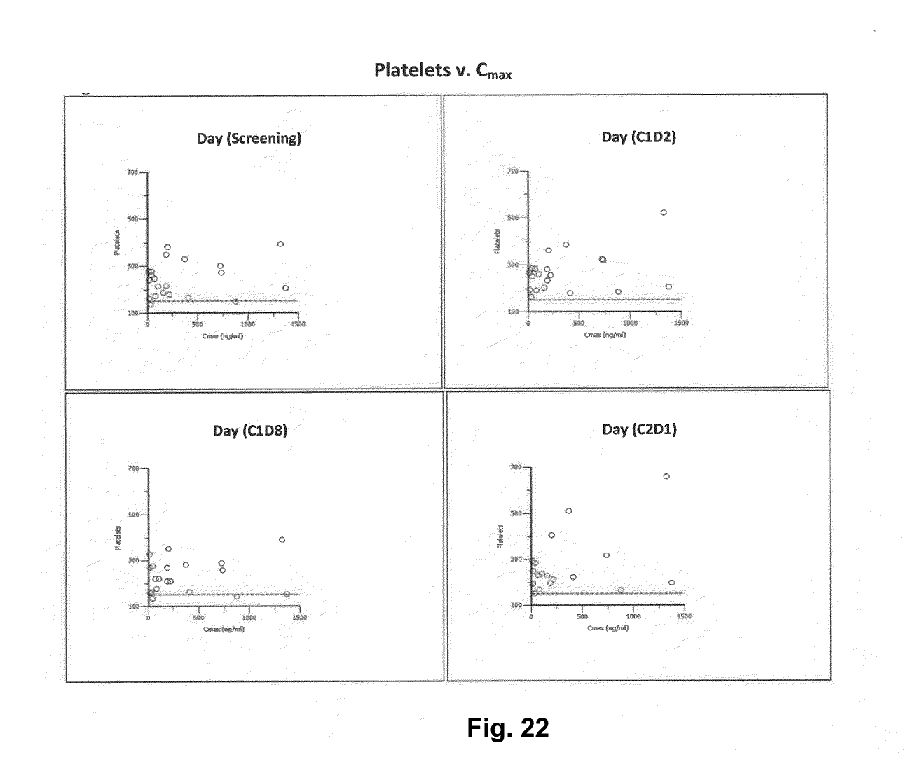

FIG. 22 shows the toxicity correlation between docetaxel Cₘₐₓ and platelets in cancer patients. Data are represented as single values.

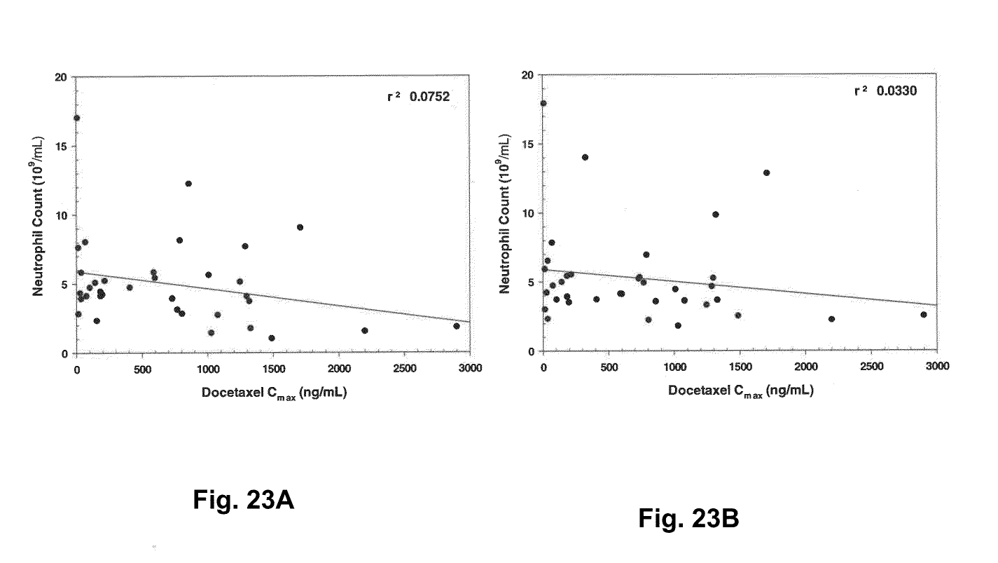

FIG. 23A shows the correlation between neutrophil count and docetaxel Cₘₐₓ in a cancer patient following a single cycle of treatment at day 8. FIG. 23B shows the correlation between neutrophil count and docetaxel Cₘₐₓ in a cancer patient following a single cycle of treatment at day 15. Data are represented as single values.

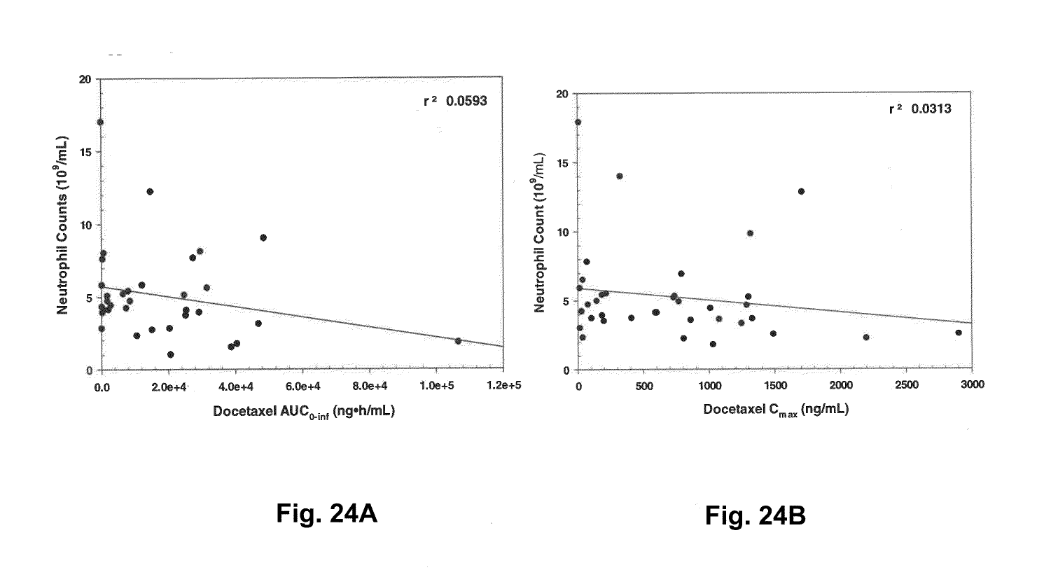

FIG. 24A shows the correlation between neutrophil count and docetaxel AUC_{0-inf} in a cancer patient following a single cycle of treatment at day 8. FIG. 24B shows the correlation between neutrophil count and docetaxel AUC_{0-inf} in a cancer patient following a single cycle of treatment at day 15. Data are represented as single values.

### DETAILED DESCRIPTION OF THE INVENTION

### I. General

The present invention provides novel liposomal taxanes, as well as a multi-step, one-pot method for encapsulation of taxanes in liposomes and subsequent incorporation of poly(ethylene glycol)-functionalized lipids into the liposomes. The liposomal taxanes prepared by the methods described herein demonstrate several advantages including increases in shelf stability, in vivo circulation time and in vivo efficacy. The liposomal taxanes are useful for the treatment of cancer as described herein.

### II. Definitions

As used herein, the term “liposome” encompasses any compartment enclosed by a lipid bilayer. The term liposome includes unilamellar vesicles which are comprised of a single lipid bilayer and generally have a diameter in the range of about 20 to about 400 nm. Liposomes can also be multilamellar, which generally have a diameter in the range of 1 to 10 μm. In some embodiments, liposomes can include multilamellar vesicles (MLVs; from about 1 μm to about 10 μm in size), large unilamellar vesicles (LUVs; from a few hundred nanometers to about 10 μm in size) and small unilamellar vesicles (SUVs; from about 20 nm to about 200 nm in size).

As used herein, the term “phosphatidylcholine lipid” refers to a diacylglyceride phospholipid having a choline headgroup (i.e., a 1,2-diacyl-sn-glycero-3-phosphocholine). The acyl groups in a phosphatidylcholine lipid are generally derived from fatty acids having from 6-24 carbon atoms. Phosphatidylcholine lipids can include synthetic and naturally-derived 1,2-diacyl-sn-glycero-3-phosphocholines.

As used herein, the term “sterol” refers to a steroid containing at least one hydroxyl group. A steroid is characterized by the presence of a fused, tetracyclic gonane ring system. Sterols include, but are not limited to, cholesterol (i.e., 2,15-dimethyl-14-(1,5-dimethylhexyl)tetracyclo[8.7.0.0^{2,7}.0^{11,15}]heptacos-7-en-5-ol; Chemical Abstracts Services Registry No. 57-88-5).

As used herein, the term “PEG-lipid” refers to a poly(ethylene glycol) polymer covalently bound to a hydrophobic or amphipilic lipid moiety. The lipid moiety can include fats, waxes, steroids, fat-soluble vitamins, monoglycerides, diglycerides, phospholipids and sphingolipids. Preferred PEG-lipids include diacyl-phosphatidylethanolamine-N-[methoxy(polyethene glycol)]s and N-acyl-sphingosine-1-{succinyl[methoxy(polyethylene glycol)]}s. The molecular weight of the PEG in the PEG-lipid is generally from about 500 to about 5000 Daltons (Da; g/mol). The PEG in the PEG-lipid can have a linear or branched structure.

As used herein, the term “taxane” refers to a compound having a structural skeleton similar to diterpene natural products, also called taxanes, initially isolated from yew trees (genus Taxus). Taxanes are generally characterized by a fused 6/8/6 tricyclic carbon backbone, and the group includes natural products and synthetic derivatives. Examples of taxanes include, but are not limited to, paclitaxel, docetaxel and cabazitaxel. Certain taxanes of the present invention include ester moieties at the 2′ hydroxyl group of the 3-phenypropionate sidechain that extends from the tricyclic taxane core.

As used herein, the term “heterocyclyl” refers to a saturated or unsaturated ring system having from 3 to 12 ring members and from 1 to 4 heteroatoms of N, O, and S. The heteroatoms can also be oxidized, such as, but not limited to, —S(O)— and —S(O)₂—. Heterocyclyl groups can include any number of ring atoms, such as, 3 to 6, 4 to 6, 5 to 6, 3 to 8, 4 to 8, 5 to 8, 6 to 8, 3 to 9, 3 to 10, 3 to 11 or 3 to 12 ring members. Any suitable number of heteroatoms can be included in the heterocyclyl groups, such as 1, 2, 3, or 4, or 1 to 2, 1 to 3, 1 to 4, 2 to 3, 2 to 4 or 3 to 4. Heterocyclyl includes, but is not limited to, 4-methylpiperazinyl, morpholino and piperidinyl.

As used herein, the term “alkanoic acid” refers to a carboxylic acid containing 2-5 carbon atoms. The alkanoic acids may be linear or branched. Examples of alkanoic acids include, but are not limited to, acetic acid, propionic acid and butanoic acid.

As used herein, the terms “molar percentage” and “mol %” refer to the number of a moles of a given lipid component of a liposome divided by the total number of moles of all lipid components. Unless explicitly stated, the amounts of active agents, diluents or other components are not included when calculating the mol % for a lipid component of a liposome.

As used herein, the term “loading” refers to effecting the accumulation of a taxane in a liposome. The taxane can be encapsulated in the aqueous interior of the liposome, or it can be embedded in the lipid bilayer. Liposomes can be passively loaded, wherein the taxane is included in the solutions used during liposome preparation. Alternatively, liposomes can be remotely loaded by establishing a chemical gradient (e.g., a pH or ion gradient) across the liposome bilayer, causing migration of the taxane from the aqueous exterior to the liposome interior.

As used herein, the term “insertion” refers to the embedding of a lipid component into a liposome bilayer. In general, an amphiphilic lipid such as a PEG-lipid is transferred from solution to the bilayer due to van der Waals interactions between the hydrophobic portion of the amphiphilic lipid and the hydrophobic interior of the bilayer.

As used herein, the term “composition” refers to a product comprising the specified ingredients in the specified amounts, as well as any product(s) which results, directly or indirectly, from the combination of the specified ingredients in the specified amounts. Pharmaceutical compositions of the present invention generally contain a liposomal taxane as described herein and a pharmaceutically acceptable carrier, diluent or excipient. By “pharmaceutically acceptable,” it is meant that the carrier, diluent or excipient must be compatible with the other ingredients of the formulation and non-deleterious to the recipient thereof.

As used herein, the term “cancer” refers to conditions including human cancers and carcinomas, sarcomas, adenocarcinomas, lymphomas, leukemias and solid and lymphoid cancers. Examples of different types of cancer include, but are not limited to, lung cancer (e.g., non-small cell lung cancer or NSCLC), ovarian cancer, prostate cancer, colorectal cancer, liver cancer (i.e., hepatocarcinoma), renal cancer (i.e., renal cell carcinoma), bladder cancer, breast cancer, thyroid cancer, pleural cancer, pancreatic cancer, uterine cancer, cervical cancer, testicular cancer, anal cancer, pancreatic cancer, bile duct cancer, gastrointestinal carcinoid tumors, esophageal cancer, gall bladder cancer, appendix cancer, small intestine cancer, stomach (gastric) cancer, cancer of the central nervous system, cancer of unknown primary origin, skin cancer, choriocarcinoma, head and neck cancer, blood cancer, osteogenic sarcoma, fibrosarcoma, neuroblastoma, glioma, melanoma, B-cell lymphoma, non-Hodgkin's lymphoma, Burkitt's lymphoma, Small Cell lymphoma, Large Cell lymphoma, monocytic leukemia, myelogenous leukemia, acute lymphocytic leukemia, acute myelocytic leukemia and multiple myeloma.

As used herein, the terms “treat”, “treating” and “treatment” refer to any indicia of success in the treatment or amelioration of a cancer or a symptom of cancer, including any objective or subjective parameter such as abatement; remission (e.g. full or partial); achieving a complete response in a patient; achieving a partial response in a patient; maintaining a stable disease state (e.g., the target lesions have not decreased in size, however, the target lesions have also not increased in size and new lesions have not formed); diminishing of symptoms or making the cancer or cancer symptom more tolerable to the patient (clinical benefit). The treatment or amelioration of symptoms can be based on any objective or subjective parameter, including, e.g., the result of a physical examination (clinical benefit) or clinical test.

As used herein, the term “full response” refers to, but is not limited to, the disappearance of all target lesions.

As used herein, the term “partial response” refers to, but is not limited to, a 30% decrease in the sum of the diameters of target lesions, taking as reference the baseline sum diameter.

As used herein, the term “progressive disease” refers to, but is not limited to, a 20% increase in the sum of the longest diameter of target lesions, taking as reference the smallest sum on study (this includes the baseline sum if that is the smallest on study) with an absolute increase of at least 5 mm and the appearance of one or more lesions.

As used herein, the term “stable disease” refers to, but is not limited to, a response that is neither sufficient to qualify for partial response nor progressive disease.

As used herein, the terms “administer”, “administered” and “administering” refer to methods of administering the liposome compositions of the present invention. The liposome compositions of the present invention can be administered in a variety of ways, including parenterally, intravenously, intradermally, intramuscularly or intraperitoneally. The liposome compositions can also be administered as part of a composition or formulation.

As used herein, the term “subject” refers to any mammal, in particular a human, at any stage of life.

The term “half-life” or “t_{1/2}.” as used herein refers to the amount of time required for the concentration or amount of the drug found in the blood or plasma to decrease by one-half. This decrease in drug concentration is a reflection of its metabolism plus excretion or elimination after absorption is complete and distribution has reached an equilibrium or quasi equilibrium state. The half-life of a drug in the blood may be determined graphically off of a pharmacokinetic plot of a drug's blood concentration-time plot, typically after intravenous administration to a sample population. The half-life can also be determined using mathematical calculations that are well known in the art. Further, as used herein, the term “half-life” also includes the “apparent half-life” of a drug. The apparent half-life may be a composite number that accounts for contributions from other processes besides elimination, such as absorption, reuptake or enterohepatic recycling.

The term “AUC” means an area under the drug concentration-time curve.

The term “Partial AUC” means an area under the drug concentration-time curve (AUC) calculated using linear trapezoidal summation for a specified interval of time, for example, AUC(0-1 hr), AUC(0-2 hr), AUC(0-4 hr), AUC(0-6 hr), AUC(0-8 hr), AUC(0-(Tmax of IR product+2SD)), AUC(0-(x)hr), AUC(x-yhr), AUC(Tmax-t), AUC(0-(t)hr), AUC(Tmax of IR product+2SD)-t) or AUC(0-∞).

The term “Cₘₐₓ” refers to the maximum plasma concentration obtain during a dosing interval.

The use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Consequently, recitation of ranges of values herein are merely intended to serve as a shorthand method of referring individually to each separate value falling within the range, unless otherwise indicated herein, and each separate value is incorporated into the specification as if it were individually recited herein.

### III. Embodiments of the Invention

In one aspect, the present invention provides a composition for the treatment of cancer. The composition includes a liposome containing a phosphatidylcholine lipid, a sterol, a PEG-lipid and a taxane or a pharmaceutically acceptable salt thereof. The taxane is esterified with a heterocyclyl-(C₂₋₅ alkanoic acid), and the PEG-lipid constitutes 2-8 mol % of the total lipids in the liposome.

In another aspect, the invention provides liposomal compositions for the treatment of cancer comprising administering to a patient in need thereof a liposome, wherein the liposome comprises: a phosphatidylcholine lipid; a sterol; a PEG-lipid; and a taxane or a pharmaceutically acceptable salt thereof; wherein the taxane is docetaxel esterified at the 2′-O-position with a heterocyclyl-(C₂₋₅ alkanoic acid); and wherein upon administration of the liposomal composition to the patient, the plasma concentration of docetaxel remain above an efficacy threshold of 0.2 μM for at least 5 hours.

### Taxanes

In some embodiments, the taxane is a compound according to Formula I, or a pharmaceutically acceptable salt thereof.

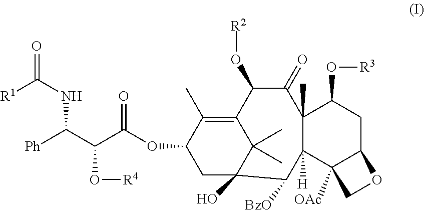

For compounds of Formula I, R¹ is selected from phenyl and t-butoxy; R² is selected from H, acetyl and methyl; R³ is selected from H, 4-(4-methylpiperazin-1-yl)-butanoyl and methyl; and R⁴ is selected from H and heterocyclyl-C₂₋₅alkanoyl. At least one of R³ and R⁴ is other than H.

Compounds of Formula I are useful as chemotherapeutic agents for the treatment of various cancers, including breast cancer, ovarian cancer and lung cancer. Formula I encompasses paclitaxel derivatives, wherein R¹ is phenyl. Paclitaxel itself can be obtained by various methods including total chemical synthesis as well as semisynthetic methods employing 10-deacetylbaccatin III (10-DAB; Formula II, below). 10-DAB can be isolated from Pacific and European yew trees (Taxus brevifolia and Taxus baccata, respectively) and can be used as a starting material for preparation of paclitaxel and other taxanes including, but not limited to, docetaxel (i.e., R¹=t-butoxy; R², R³, R⁴=H) and cabazitaxel according to known methods. Taxane preparation via semisynthetic methods are contemplated for use in the present invention in addition to taxane preparation via total synthesis.

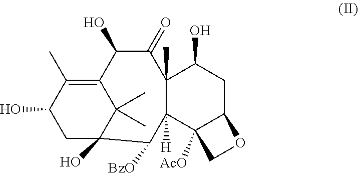

As described above, the use of taxanes—including paclitaxel and docetaxel—for cancer therapy can be limited by low bioavailability due to inadequate solubility, as well as by high toxicity. Various strategies have been employed to remedy these drawbacks. For example, derivatization of the taxane skeleton at the C7 and C10 functional groups of the tricyclic core, or at the C2′ hydroxyl group of the C13 sidechain, with moieties of varying polarity can be used to alter the bioavailability of taxane-base drugs (see, e.g., U.S. Pat. No. 6,482,850; U.S. Pat. No. 6,541,508; U.S. Pat. No. 5,608,087 and U.S. Pat. No. 5,824,701).

Incorporation of a taxane into liposomes can improve bioavailability and reduce the toxicity of the taxane. In the present invention, modification of the taxane skeleton with weak base moieties can facilitate the active loading of otherwise poorly water-soluble taxanes into the aqueous interior of a liposome. In general, the weak base moiety can include an ionizable amino group, such as an N-methyl-piperazino group, a morpholino group, a piperidino group, a bis-piperidino group or a dimethylamino group. In some embodiments, the weak base moiety is an N-methyl-piperazino group.

A taxane can be derivatized in a region that is not essential for the intended therapeutic activity such that the activity of the derivative is substantially equivalent to that of the free drug. For example, in some aspects, the weak base derivative comprises the taxane docetaxel derivatized at the 7-OH group of the baccatin skeleton. In some embodiments, docetaxel derivatives are provided that are derivatized at the 2′-OH group, which is essential for docetaxel activity.

In some embodiments, the taxane derivative has the following formula:

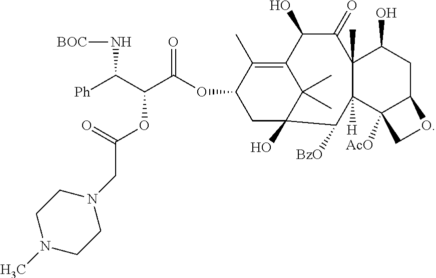

(hereinafter, “TD-1”). In other embodiments, the taxane derivative is a pharmaceutically acceptable salt of TD-1.

Accordingly, some embodiments of the present invention provide liposomes containing a taxane or a pharmaceutically acceptable salt thereof, wherein the taxane is docetaxel esterified at the 2′-O-position with a heterocyclyl-(C₂₋₅alkanoic acid) (i.e., the taxane is a compound of Formula I wherein R¹ is t-butoxy; R² is H; R³ is H; and R⁴ is heterocyclyl-C₂₋₅alkanoyl). In some embodiments, the heterocyclyl-(C₂₋₅alkanoic acid) is selected from 5-(4-methylpiperazin-1-yl)-pentanoic acid, 4-(4-methylpiperazin-1-yl)-butanoic acid, 3-(4-methylpiperazin-1-yl)-propionic acid, 2-(4-methylpiperazin-1-yl)-ethanoic acid, 5-morpholino-pentanoic acid, 4-morpholino-butanoic acid, 3-morpholino-propionic acid, 2-morpholino-ethanoic acid, 5-(piperidin-1-yl)pentanoic acid, 4-(piperidin-1-yl)butanoic acid, 3-(piperidin-1-yl)propionic acid and 2-(piperidin-1-yl) ethanoic acid. In some embodiments, the heterocyclyl-(C₂₋₅alkanoic acid) is 4-(4-methylpiperazin-1-yl)-butanoic acid.

### Liposomes

The liposomes of the present invention can contain any suitable lipid, including cationic lipids, zwitterionic lipids, neutral lipids or anionic lipids as described above. Suitable lipids can include fats, waxes, steroids, cholesterol, fat-soluble vitamins, monoglycerides, diglycerides, phospholipids, sphingolipids, glycolipids, cationic or anionic lipids, derivatized lipids, and the like.

In general, the liposomes of the present invention contain at least one phosphatidylcholine (PC) lipid. Suitable PC lipids include saturated PCs and unsaturated PCs.

Examples of saturated PCs include 1,2-dilauroyl-sn-glycero-3-phosphocholine (DLPC), 1,2-dimyristoyl-sn-glycero-3-phosphocholine (dimyristoylphosphatidylcholine; DMPC), 1,2-distearoyl-sn-glycero-3-phosphocholine (distearoylphosphatidyl choline; DSPC), 1,2-dioleoyl-sn-glycero-3-phosphocholine (DOPC), 1,2-dipalmitoyl-sn-glycero-3-phosphocholine (dipalmitoylphosphatidylcholine; DPPC), 1-myristoyl-2-palmitoyl-sn-glycero-3-phosphocholine (MPPC), 1-palmitoyl-2-myristoyl-sn-glycero-3-phosphocholine (PMPC), 1-myristoyl-2-stearoyl-sn-glycero-3-phosphocholine (MSPC), 1-palmitoyl-2-stearoyl-sn-glycero-3-phosphocholine (PSPC), 1-stearoyl-2-palmitoyl-sn-glycero-3-phosphocholine (SPPC) and 1-stearoyl-2-myristoyl-sn-glycero-3-phosphocholine (SMPC).

Examples of unsaturated PCs include, but are not limited to, 1,2-dimyristoleoyl-sn-glycero-3-phosphocholine, 1,2-dimyristelaidoyl-sn-glycero-3-phosphocholine, 1,2-dipamiltoleoyl-sn-glycero-3-phosphocholine, 1,2-dipalmitelaidoyl-sn-glycero-3-phosphocholine, 1,2-dioleoyl-sn-glycero-3-phosphocholine (DOPC), 1,2-dielaidoyl-sn-glycero-3-phosphocholine, 1,2-dipetroselenoyl-sn-glycero-3-phosphocholine, 1,2-dilinoleoyl-sn-glycero-3-phosphocholine, 1-palmitoyl-2-oleoyl-sn-glycero-3-phosphocholine (palmitoyloleoylphosphatidylcholine) POPC), 1-palmitoyl-2-linoleoyl-sn-glycero-3-phosphocholine, 1-stearoyl-2-oleoyl-sn-glycero-3-phosphocholine (SOPC), 1-stearoyl-2-linoleoyl-sn-glycero-3-phosphocholine, 1-oleoyl-2-myristoyl-sn-glycero-3-phosphocholine (OMPC), 1-oleoyl-2-palmitoyl-sn-glycero-3-phosphocholine (OPPC) and 1-oleoyl-2-stearoyl-sn-glycero-3-phosphocholine (OSPC).

Lipid extracts, such as egg PC, heart extract, brain extract, liver extract, soy PC and hydrogenated soy PC (HSPC) are also useful in the present invention.

The liposomal formulations provided herein will, in some embodiments, consist essentially of PC/cholesterol mixtures (with an added taxane and PEG-lipid as described below). In some embodiments, the liposomal formulations will consist essentially of a phosphatidylcholine lipid or mixture of phosphatidylcholine lipids, with cholesterol, a PEG-lipid and a taxane. In still other embodiments, the liposomal formulations will consist essentially of a single type of phosphatidylcholine lipid, with cholesterol, a PEG-lipid and a taxane. In some embodiments, when a single type of phosphatidylcholine lipid is used, it is selected from the group consisting of: DOPC, DSPC, HSPC, DPPC, POPC and SOPC.

In some embodiments, the phosphatidylcholine lipid is selected from the group consisting of DPPC, DSPC, HSPC and mixtures thereof. In some embodiments, the liposomal formulations of the present invention include liposomes containing about 45 to about 70 mol % of a phosphatidylcholine lipid or mixture of phosphatidylcholine lipids, about 50 to about 65 mol % of a phosphatidylcholine lipid or mixture of phosphatidylcholine lipids, about 50 to about 56 mol % of a phosphatidylcholine lipid or mixture of phosphatidylcholine lipids, or about 53 to about 56 mol % of a phosphatidylcholine lipid or mixture of phosphatidylcholine lipids. The liposomes can contain, for example, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69 or about 70 mol % phosphatidylcholine or a mixture thereof. In some embodiments, the liposomes contain about 65 mol % phosphatidylcholine or a mixture thereof. In other embodiments, the liposomes contain about 60 mol % phosphatidylcholine or a mixture thereof. In still other embodiments, the liposomes contain about 56 mol % phosphatidylcholine or a mixture thereof. In other embodiments, the liposomes contain about 55 mol % phosphatidylcholine or a mixture thereof. In additional embodiments, the liposomes contain about 54 mol % phosphatidylcholine or a mixture thereof. In further embodiments, the liposomes contain about 53 mol % phosphatidylcholine or a mixture thereof. In still further embodiments, the liposomes contain about 52 mol % phosphatidylcholine or a mixture thereof. In other embodiments, the liposomes contain about 51 mol % phosphatidylcholine or a mixture thereof. In further embodiments, the liposomes contain about 50 mol % phosphatidylcholine or a mixture thereof.

The liposomes can contain, for example, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69 or about 70 mol % phosphatidylcholine. In some embodiments, the liposomes contain about 65 mol % phosphatidylcholine. In other embodiments, the liposomes contain about 60 mol % phosphatidylcholine. In still other embodiments, the liposomes contain about 56 mol % phosphatidylcholine. In other embodiments, the liposomes contain about 55 mol % phosphatidylcholine. In additional embodiments, the liposomes contain about 54 mol % phosphatidylcholine. In further embodiments, the liposomes contain about 53 mol % phosphatidylcholine. In still further embodiments, the liposomes contain about 52 mol % phosphatidylcholine. In other embodiments, the liposomes contain about 51 mol % phosphatidylcholine. In further embodiments, the liposomes contain about 50 mol % phosphatidylcholine.

Other suitable phospholipids, generally used in low amounts or in amounts less than the phosphatidylcholine lipids, include phosphatidic acids (PAs), phosphatidylethanolamines (PEs), phosphatidylglycerols (PGs), phosphatidylserine (PSs), and phosphatidylinositol (PIs). Examples of phospholipids include, but are not limited to, dimyristoylphosphatidylglycerol (DMPG), distearoylphosphatidylglycerol (DSPG), dioleoylphosphatidylglycerol (DOPG), dipalmitoylphosphatidylglycerol (DPPG), dimyristoylphosphatidylserine (DMP 5), distearoylphosphatidylserine (DSPS), dioleoylphosphatidylserine (DOPS), dipalmitoylphosphatidylserine (DPPS), dioleoylphosphatidylethanolamine (DOPE), POPC; palmitoyloleoylphosphatidylethanolamine (POPE), dipalmitoylphosphatidylethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearoyl-2-oleoyl-phosphatidyethanolamine (SOPE), dielaidoylphosphoethanolamine (transDOPE) and cardiolipin.

In some embodiments, phospholipids can include reactive functional groups for further derivatization. Examples of such reactive lipids include, but are not limited to, dioleoylphosphatidylethanolamine-4-(N-maleimidomethyl)-cyclohexane-1-carboxylate (DOPE-mal) and dipalmitoylphosphatidylethanolamine-N-succinyl (succinyl-PE).

Liposomes of the present invention can contain steroids, characterized by the presence of a fused, tetracyclic gonane ring system. Examples of steroids include, but are not limited to, cholic acid, progesterone, cortisone, aldosterone, testosterone, dehydroepiandrosterone and sterols, such as estradiol and cholesterol. Synthetic steroids and derivatives thereof are also contemplated for use in the present invention.

In general, the liposomes contain at least one sterol. In some embodiments, the sterol is cholesterol (i.e., 2,15-dimethyl-14-(1,5-dim ethylhexyl)tetracyclo[8.0.7.0.0^{2,7}.0^{01,15}]heptacos-7-en-5-01). In some embodiments, the liposomes can contain about 30-50 mol % of cholesterol or about 30-45 mol % of cholesterol. The liposomes can contain, for example, about 30, about 31, about 32, about 33, about 34, about 35, about 36, about 37, about 38, about 39, about 40, about 41, about 42, about 43, about 44, about 45, about 46, about 47, about 48, about 49 or about 50 mol % cholesterol. In some embodiments, the liposomes contain about 30 to about 40 mol % cholesterol. In some embodiments, the liposomes contain about 40 to about 45 mol % cholesterol. In some embodiments, the liposomes contain about 45 mol % cholesterol. In some embodiments, the liposomes contain about 44 mol % cholesterol. In other embodiments, the liposomes contain about 40 mol % cholesterol. In other embodiments, the liposomes contain about 35 mol % cholesterol. In further embodiments, the liposomes contain about 30 mol % cholesterol.

The liposomes of the present invention can include any suitable poly(ethylene glycol)-lipid derivative (PEG-lipid). In some embodiments, the PEG-lipid is a diacyl-phosphatidylethanolamine-N-[methoxy(polyethene glycol)]. The molecular weight of the poly(ethylene glycol) in the PEG-lipid is generally in the range of from about 500 Da to about 5000 Da. The poly(ethylene glycol) can have a molecular weight of, for example, 750 Da, 1000 Da, 2000 Da or 5000 Da. In some embodiments, the PEG-lipid is selected from distearoyl-phosphatidylethanolamine-N-[methoxy(polyethene glycol)-2000] (DSPE-PEG-2000) and distearoyl-phosphatidylethanolamine-N-[methoxy(polyethene glycol)-5000] (DSPE-PEG-5000). In some embodiments, the PEG-lipid is DSPE-PEG-2000.

In general, the compositions of the present invention include liposomes containing about 2 to about 8 mol % of the PEG-lipid. The liposomes can contain, for example, about 2, about 3, about 4, about 5, about 6, about 7 or about 8 mol % PEG-lipid. In some embodiments, the liposomes contain about 2 to about 6 mol % PEG-lipid. In some embodiments, the liposomes contain about 5 mol % PEG-lipid. In other embodiments, the liposomes contain about 3 mol % PEG-lipid. In some embodiments, the liposomes contain about 3 mol % DSPE-PEG-2000.

The liposomes of the present invention can also include some amounts of cationic lipids, which are generally in amounts lower than the amount of phosphatidylcholine lipid. Cationic lipids contain positively charged functional groups under physiological conditions. Cationic lipids include, but are not limited to, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N-[1-(2,3,-ditetradecyloxy)propyl]-N,N-dimethyl-N-hydroxyethylammonium bromide (DMRIE), N-[1-(2,3,dioleyloxy)propyl]-N,N-dimethyl-N-hydroxy ethylammonium bromide (DORIE), 3β-[N—(N′,N′-dimethylaminoethane) carbamoyl]cholesterol (DC-Chol), dimethyldioctadecylammonium (DDAB) and N,N-dimethyl-2,3-dioleyloxy)propylamine (DODMA).

In some embodiments of the present invention, the liposome includes from about 50 mol % to about 70 mol % of DSPC and from about 25 mol % to about 45 mol % of cholesterol. In some embodiments, the liposome includes about 53 mol % of DSPC, about 44 mol % of cholesterol and about 3 mol % of DSPE-PEG-2000. In some embodiments, the liposome includes about 66 mol % of DSPC, about 30 mol % of cholesterol and about 4 mol % of DSPE-PEG-2000.

In further embodiments, the liposome includes about 50 mol % of DSPC, about 45 mol % of cholesterol and about 5 mol % of DSPE-PEG-2000; about 55 mol % of DSPC, about 40 mol % of cholesterol and about 5 mol % of DSPE-PEG-2000; about 60 mol % of DSPC, about 35 mol % of cholesterol and about 5 mol % of DSPE-PEG-2000; about 65 mol % of DSPC, about 30 mol % of cholesterol and about 5 mol % of DSPE-PEG-2000; and about 70 mol % of DSPC, about 25 mol % of cholesterol and about 5 mol % of DSPE-PEG-2000.

### Diagnostic Agents

The liposomes of the present invention may also contain diagnostic agents. A diagnostic agent used in the present invention can include any diagnostic agent known in the art, as provided, for example, in the following references: Armstrong et al., Diagnostic Imaging, 5th Ed., Blackwell Publishing (2004); Torchilin, V. P., Ed., Targeted Delivery of Imaging Agents, CRC Press (1995); Vallabhajosula, S., Molecular Imaging: Radiopharmaceuticals for PET and SPECT, Springer (2009). A diagnostic agent can be detected by a variety of ways, including as an agent providing and/or enhancing a detectable signal that includes, but is not limited to, gamma-emitting, radioactive, echogenic, optical, fluorescent, absorptive, magnetic or tomography signals. Techniques for imaging the diagnostic agent can include, but are not limited to, single photon emission computed tomography (SPECT), magnetic resonance imaging (MRI), optical imaging, positron emission tomography (PET), computed tomography (CT), x-ray imaging, gamma ray imaging, and the like. The diagnostic agents can be associated with the therapeutic liposome in a variety of ways, including for example being embedded to or encapsulated in the liposome.

In some embodiments, a diagnostic agent can include chelators that bind to metal ions to be used for a variety of diagnostic imaging techniques. Exemplary chelators include, but are not limited to, ethylenediaminetetraacetic acid (EDTA), [4-(1,4,8, 11-tetraazacyclotetradec-1-yl) methyl]benzoic acid (CPTA), cyclohexanediaminetetraacetic acid (CDTA), ethylenebis(oxyethylenenitrilo)tetraacetic acid (EGTA), diethylenetriaminepentaacetic acid (DTPA), citric acid, hydroxyethyl ethylenediamine triacetic acid (HEDTA), iminodiacetic acid (IDA), triethylene tetraamine hexaacetic acid (TTHA), 1,4,7, 10-tetraazacyclododecane-1,4,7,10-tetra(m ethylene phosphonic acid) (DOTP), 1,4,8,11-tetraazacyclododecane-1,4,8,11-tetraacetic acid (TETA), 1,4,7,10-tetraazacyclododecane-1,4,7,10-tetraacetic acid (DOTA) and derivatives thereof.

A radioisotope can be incorporated into some of the diagnostic agents described herein and can include radionuclides that emit gamma rays, positrons, beta and alpha particles, and X-rays. Suitable radionuclides include, but are not limited to, ²²⁵Ac, ⁷²As, ²¹¹ At, ¹¹B, ¹²⁸Ba, ²¹²Bi, ⁷⁵Br, ⁷⁷Br, ¹⁴C, ¹⁰⁹Cd, ⁶²Cu, ⁶⁴Cu, ⁶⁷Cu, ¹⁸F, ⁶⁷Ga, ⁶⁸Ga, ³H, ¹²³I, ¹²⁵I, ¹³⁰I, ¹³¹I, ¹¹¹In, ¹⁷⁷Lu, ¹³N, ¹⁵O, ³²P, ³³P, ²¹²Pb, ¹⁰³Pd, ¹⁸⁶Re, ¹⁸⁸Re, ⁴⁷Sc, ¹⁵³Sm, ⁸⁹Sr, ^{99m}Tc, ⁸⁸Y and ⁹⁰Y. In certain embodiments, radioactive agents can include ¹¹¹In-DTPA, ^{99m}Tc(CO)₃-DTPA, ^{99m}Tc(CO)₃-ENpy2, ^{62/64/67}Cu-TETA, ^{99m}Tc(CO)₃-IDA and ^{99m}Tc(CO)₃triamines (cyclic or linear). In other embodiments, the agents can include DOTA and its various analogs with ¹¹¹In, ¹⁷⁷Lu, ¹⁵³Sm, ^{88/90}Y, ^{62/64/67}Cu or ^{67/68}Ga. In some embodiments, the liposomes can be radiolabeled, for example, by incorporation of lipids attached to chelates, such as DTPA-lipid, as provided in the following references: Phillips et al., Wiley Interdisciplinary Reviews: Nanomedicine and Nanobiotechnology, 1(1): 69-83 (2008); Torchilin, V. P. & Weissig, V., Eds. Liposomes 2nd Ed.: Oxford Univ. Press (2003); Elbayoumi, T. A. & Torchilin, V. P., Eur. J. Nucl. Med. Mol. Imaging 33:1196-1205 (2006); Mougin-Degraef, M. et al., Int'l J. Pharmaceutics 344:110-117 (2007).

In other embodiments, the diagnostic agents can include optical agents such as fluorescent agents, phosphorescent agents, chemiluminescent agents, and the like. Numerous agents (e.g., dyes, probes, labels, or indicators) are known in the art and can be used in the present invention. (See, e.g., Invitrogen, The Handbook—A Guide to Fluorescent Probes and Labeling Technologies, Tenth Edition (2005)). Fluorescent agents can include a variety of organic and/or inorganic small molecules or a variety of fluorescent proteins and derivatives thereof. For example, fluorescent agents can include, but are not limited to, cyanines, phthalocyanines, porphyrins, indocyanines, rhodamines, phenoxazines, phenylxanthenes, phenothiazines, phenoselenazines, fluoresceins, benzoporphyrins, squaraines, dipyrrolo pyrimidones, tetracenes, quinolines, pyrazines, corrins, croconiums, acridones, phenanthridines, rhodamines, acridines, anthraquinones, chalcogenopyrylium analogues, chlorins, naphthalocyanines, methine dyes, indolenium dyes, azo compounds, azulenes, azaazulenes, triphenyl methane dyes, indoles, benzoindoles, indocarbocyanines, benzoindocarbocyanines, and BODIPY™ derivatives having the general structure of 4,4-difluoro-4-bora-3a,4a-diaza-s-indacene, and/or conjugates and/or derivatives of any of these. Other agents that can be used include, but are not limited to, for example, fluorescein, fluorescein-polyaspartic acid conjugates, fluorescein-polyglutamic acid conjugates, fluorescein-polyarginine conjugates, indocyanine green, indocyanine-dodecaaspartic acid conjugates, indocyanine-polyaspartic acid conjugates, isosulfan blue, indole disulfonates, benzoindole disulfonate, bis(ethylcarboxymethyl)indocyanine, bis(pentylcarboxymethyl)indocyanine, polyhydroxyindole sulfonates, polyhydroxyb enzoindole sulfonate, rigid heteroatomic indole sulfonate, indocyaninebispropanoic acid, indocyaninebishexanoic acid, 3,6-dicyano-2,5-[(N,N,N′,N′-tetrakis(carboxymethyl)amino]pyrazine, 3,6-[(N,N,N′,N′-tetrakis(2-hydroxyethyl)amino]pyrazine-2, 5-dicarboxylic acid, 3,6-bis(N-azatedino)pyrazine-2, 5-dicarboxylic acid, 3,6-bis(N-morpholino)pyrazine-2,5-dicarboxylic acid, 3,6-bis(N-piperazino)pyrazine-2, 5-dicarboxylic acid, 3,6-bis(N-thiomorpholino)pyrazine-2, 5-dicarboxylic acid, 3,6-bis(N-thiomorpholino)pyrazine-2, 5-dicarboxylic acid S-oxide, 2,5-dicyano-3,6-bis(N-thiomorpholino)pyrazine S,S-dioxide, indocarbocyaninetetrasulfonate, chloroindocarbocyanine and 3,6-diaminopyrazine-2,5-dicarboxylic acid.

One of ordinary skill in the art will appreciate that particular optical agents used can depend on the wavelength used for excitation, depth underneath skin tissue and other factors generally well known in the art. For example, optimal absorption or excitation maxima for the optical agents can vary depending on the agent employed, but in general, the optical agents of the present invention will absorb or be excited by light in the ultraviolet (UV), visible or infrared (IR) range of the electromagnetic spectrum. For imaging, dyes that absorb and emit in the near-IR (˜700-900 nm, e.g., indocyanines) are preferred. For topical visualization using an endoscopic method, any dyes absorbing in the visible range are suitable.

In some embodiments, the non-ionizing radiation employed in the process of the present invention can range in wavelength from about 350 nm to about 1200 nm. In one exemplary embodiment, the fluorescent agent can be excited by light having a wavelength in the blue range of the visible portion of the electromagnetic spectrum (from about 430 nm to about 500 nm) and emits at a wavelength in the green range of the visible portion of the electromagnetic spectrum (from about 520 nm to about 565 nm). For example, fluorescein dyes can be excited with light with a wavelength of about 488 nm and have an emission wavelength of about 520 nm. As another example, 3,6-diaminopyrazine-2,5-dicarboxylic acid can be excited with light having a wavelength of about 470 nm and fluoresces at a wavelength of about 532 nm. In another embodiment, the excitation and emission wavelengths of the optical agent may fall in the near-infrared range of the electromagnetic spectrum. For example, indocyanine dyes, such as indocyanine green, can be excited with light at a wavelength of about 780 nm and have an emission wavelength of about 830 nm.

In yet other embodiments, the diagnostic agents can include, but are not limited to, magnetic resonance (MR) and x-ray contrast agents that are generally well known in the art, including, for example, iodine-based x-ray contrast agents, superparamagnetic iron oxide (SPIO), complexes of gadolinium or manganese, and the like. (See, e.g., Armstrong et al., Diagnostic Imaging, 5th Ed., Blackwell Publishing (2004)). In some embodiments, a diagnostic agent can include a MR imaging agent. Exemplary MR agents include, but are not limited to, paramagnetic agents, superparamagnetic agents, and the like. Exemplary paramagnetic agents can include, but are not limited to, gadopentetic acid, gadoteric acid, gadodiamide, gadolinium, gadoteridol, mangafodipir, gadoversetamide, ferric ammonium citrate, gadobenic acid, gadobutrol and gadoxetic acid. Superparamagnetic agents can include, but are not limited to, superparamagnetic iron oxide and ferristene. In certain embodiments, the diagnostic agents can include x-ray contrast agents as provided, for example, in the following references: H. S Thomsen, R. N. Muller and R. F. Mattrey, Eds., Trends in Contrast Media, (Berlin: Springer-Verlag, 1999); P. Dawson, D. Cosgrove and R. Grainger, Eds., Textbook of Contrast Media (ISIS Medical Media 1999); Torchilin, V. P., Curr. Pharm. Biotech. 1:183-215 (2000); Bogdanov, A. A. et al., Adv. Drug Del. Rev. 37:279-293 (1999); Sachse, A. et al., Investigative Radiology 32(1):44-50 (1997). Examples of x-ray contrast agents include, without limitation, iopamidol, iomeprol, iohexol, iopentol, iopromide, iosimide, ioversol, iotrolan, iotasul, iodixanol, iodecimol, ioglucamide, ioglunide, iogulamide, iosarcol, ioxilan, iopamiron, metrizamide, iobitridol and iosimenol. In certain embodiments, the x-ray contrast agents can include iopamidol, iomeprol, iopromide, iohexol, iopentol, ioversol, iobitridol, iodixanol, iotrolan, and iosimenol.

### Targeting Agents

In some cases, liposome accumulation at a target site may be due to the enhanced permeability and retention characteristics of certain tissues such as cancer tissues. Accumulation in such a manner often results in part because of liposome size and may not require special targeting functionality. In other cases, the liposomes of the present invention can also include a targeting agent. Generally, the targeting agents of the present invention can associate with any target of interest, such as a target associated with an organ, tissues, cell, extracellular matrix or intracellular region. In certain embodiments, a target can be associated with a particular disease state, such as a cancerous condition. In some embodiments, the targeting component can be specific to only one target, such as a receptor. Suitable targets can include, but are not limited to, a nucleic acid, such as a DNA, RNA, or modified derivatives thereof. Suitable targets can also include, but are not limited to, a protein, such as an extracellular protein, a receptor, a cell surface receptor, a tumor-marker, a transmembrane protein, an enzyme or an antibody. Suitable targets can include a carbohydrate, such as a monosaccharide, disaccharide or polysaccharide that can be, for example, present on the surface of a cell.

In certain embodiments, a targeting agent can include a target ligand (e.g., an RGD-containing peptide), a small molecule mimic of a target ligand (e.g., a peptide mimetic ligand) or an antibody or antibody fragment specific for a particular target. In some embodiments, a targeting agent can further include folic acid derivatives, B-12 derivatives, integrin RGD peptides, NGR derivatives, somatostatin derivatives or peptides that bind to the somatostatin receptor, e.g., octreotide and octreotate, and the like. The targeting agents of the present invention can also include an aptamer. Aptamers can be designed to associate with or bind to a target of interest. Aptamers can be comprised of, for example, DNA, RNA and/or peptides, and certain aspects of aptamers are well known in the art. (See. e.g., Klussman, S., Ed., The Aptamer Handbook, Wiley-VCH (2006); Nissenbaum, E. T., Trends in Biotech. 26(8): 442-449 (2008)).

### Methods for Preparing Liposomal Taxane

In another aspect, the invention provides methods for preparing a liposomal taxane. Liposomes can be prepared and loaded with taxanes using a number of techniques that are known to those of skill in the art. Lipid vesicles can be prepared, for example, by hydrating a dried lipid film (prepared via evaporation of a mixture of the lipid and an organic solvent in a suitable vessel) with water or an aqueous buffer. Hydration of lipid films typically results in a suspension of multilamellar vesicles (MLVs). Alternatively, MLVs can be formed by diluting a solution of a lipid in a suitable solvent, such as a C₁₋₄ alkanol, with water or an aqueous buffer. Unilamellar vesicles can be formed from MLVs via sonication or extrusion through membranes with defined pore sizes. Encapsulation of a taxane can be conducted by including the drug in the aqueous solution used for film hydration or lipid dilution during MLV formation. Taxanes can also be encapsulated in pre-formed vesicles using “remote loading” techniques. Remote loading includes the establishment of a pH- or ion-gradient on either side of the vesicle membrane, which drives the taxane from the exterior solution to the interior of the vesicle.

Accordingly, some embodiments of the present invention provide a method for preparing a liposomal taxane including: a) forming a first liposome having a lipid bilayer including a phosphatidylcholine lipid and a sterol, wherein the lipid bilayer encapsulates an interior containing an aqueous solution; b) loading the first liposome with a taxane, or a pharmaceutically acceptable salt thereof, to form a loaded liposome, wherein the taxane is docetaxel esterified at the 2′-O-position with a heterocyclyl-(C₂₋₅alkanoyl) group; and c) incorporating the PEG-lipid into the lipid bilayer.

In another embodiment, the present invention provides a method for preparing a liposomal taxane including: a) forming a first liposome having a lipid bilayer including a phosphatidylcholine lipid, a sterol and a PEG-lipid, wherein the lipid bilayer encapsulates an interior containing an aqueous solution; and b) loading the first liposome with a taxane, or a pharmaceutically acceptable salt thereof, to form a loaded liposome, wherein the taxane is docetaxel esterified at the 2′-O-position with a heterocyclyl-(C₂₋₅alkanoyl) group.

The taxanes and lipids used in the methods of the invention are generally as described above. However, the route to the liposomal taxane will depend in part on the identity of the specific taxane and lipids, and the quantities and combinations that are used. For example, the taxane can be encapsulated in vesicles at various stages of liposome preparation. In some embodiments, the first liposome is formed such that the lipid bilayer comprises DSPC and cholesterol, and the DSPC:cholesterol ratio is about 55:45 (mol:mol). In some embodiments, the first liposome is formed such that the lipid bilayer comprises DSPC and cholesterol, and the DSPC:cholesterol ratio is about 70:30 (mol:mol). In some embodiments, the interior of the first liposome contains aqueous ammonium sulfate buffer. Loading the first liposomes can include forming an aqueous solution containing the first liposome and the taxane or pharmaceutically acceptable salt thereof under conditions sufficient to allow accumulation of the taxane in the interior compartment of the first liposome.

Loading conditions generally include a higher ammonium sulfate concentration in the interior of the first liposome than in the exterior aqueous solution. In some embodiments, the loading step is conducted at a temperature above the gel-to-fluid phase transition temperature (Tₘ) of one or more of the lipid components in the liposomes. The loading can be conducted, for example, at about 50° C., about 55° C., about 60° C., about 65° C. or at about 70° C. In some embodiments, the loading step is conducted at a temperature of from about 50° C. to about 70° C. Loading can be conducted using any suitable amount of the taxane. In general, the taxane is used in an amount such that the ratio of the combined weight of the phosphatidylcholine and the sterol in the liposome to the weight of the taxane is from about 1:0.01 to about 1:1. The ratio of the combined phosphatidylcholine/sterol to the weight of the taxane can be, for example, about 1:0.01, about 1:0.05, about 1:0.10, about 1:0.15, about 1:0.20, about 1:0.25, about 1:0.30, about 1:0.35, about 1:0.40, about 1:0.45, about 1:0.50, about 1:0.55, about 1:0.60, about 1:0.65, about 1:0.70, about 1:0.75, about 1:0.80, about 1:0.85, about 1:0.90, about 1:0.95 or about 1:1. In some embodiments, the loading step is conducted such that the ratio of the combined weight of the phosphatidylcholine and the sterol to the weight of the taxane is from about 1:0.01 to about 1:1. In some embodiments, the ratio of the combined weight of the phosphatidylcholine and the sterol to the weight of the taxane is from about 1:0.05 to about 1:0.5. In some embodiments, the ratio of the combined weight of the phosphatidylcholine and the sterol to the weight of the taxane is about 1:0.2. The loading step can be conducted for any amount of time that is sufficient to allow accumulation of the taxane in the liposome interior at a desired level.

The PEG-lipid can also be incorporated into lipid vesicles at various stages of the liposome preparation. For example, MLVs containing a PEG-lipid can be prepared prior to loading with a taxane. Alternatively, a PEG-lipid can be inserted into a lipid bilayer after loading of a vesicle with a taxane. The PEG-lipid can be inserted into MLVs prior to extrusion of SUVs, or the PEG-lipid can be inserted into pre-formed SUVs.

Accordingly, some embodiments of the invention provide a method for preparing a liposomal taxane wherein the method includes: a) forming a first liposome having a lipid bilayer including a phosphatidylcholine lipid and a sterol, wherein the lipid bilayer encapsulates an interior compartment comprising an aqueous solution; b) loading the first liposome with a taxane, or a pharmaceutically acceptable salt thereof, to form a loaded liposome, wherein the taxane is docetaxel esterified at the 2′-O-position with a heterocyclyl-(C₂₋₅alkanoyl) group; and c) forming a mixture containing the loaded liposome and a poly(ethylene glycol)-phospholipid conjugate (PEG-lipid) under conditions sufficient to allow insertion of the PEG-lipid into the lipid bilayer.

In some embodiments, the insertion of the PEG-lipid is conducted at a temperature of from about 35 to about 70° C. The loading can be conducted, for example, at about 35° C., about 40° C., about 45° C., about 50° C., about 55° C., about 60° C., about 65° C. or at about 70° C. In some embodiments, insertion of the PEG-lipid is conducted at a temperature of from about 50° C. to about 55° C. Insertion can be conducted using any suitable amount of the PEG-lipid. In general, the PEG-lipid is used in an amount such that the ratio of the combined number of moles of the phosphatidylcholine and the sterol to the number of moles of the PEG-lipid is from about 1000:1 to about 20:1. The molar ratio of the combined phosphatidylcholine/sterol to PEG lipid can be, for example, about 1000:1, about 950:1, about 900:1, about 850:1, about 800:1, about 750:1, about 700:1, about 650:1, about 600:1, about 550:1, about 500:1, about 450:1, about 400:1, about 350:1, about 300:1, about 250:1, about 200:1, about 150:1, about 100:1, about 50:1 or about 20:1. In some embodiments, the loading step is conducted such that the ratio of combined phosphatidylcholine and sterol to PEG-lipid is from about 1000:1 to about 20:1 (mol:mol). In some embodiments, the ratio of the combined phosphatidylcholine and sterol to the PEG-lipid is from about 100:1 to about 20:1 (mol:mol). In some embodiments, the ratio of the combined phosphatidylcholine and sterol to the PEG-lipid is from about 35:1 (mol:mol) to about 25:1 (mol:mol). In some embodiments, the ratio of the combined phosphatidylcholine and sterol to the PEG-lipid is about 33:1 (mol:mol). In some embodiments, the ratio of the combined phosphatidylcholine and sterol to the PEG-lipid is about 27:1 (mol:mol).

A number of additional preparative techniques known to those of skill in the art can be included in the methods of the invention. Liposomes can be exchanged into various buffers by techniques including dialysis, size exclusion chromatography, diafiltration and ultrafiltration. Buffer exchange can be used to remove unencapsulated taxanes and other unwanted soluble materials from the compositions. Aqueous buffers and certain organic solvents can be removed from the liposomes via lyophilization. In some embodiments, the methods of the invention include exchanging the liposomal taxane from the mixture in step c) to an aqueous solution that is substantially free of unencapsulated taxane and uninserted PEG-lipid. In some embodiments, the methods include lyophilizing the liposomal taxane.

### Methods of Treating Cancer

In another aspect, the invention provides a method of treating cancer. The method includes administering to a subject in need thereof a pharmaceutical composition containing a liposomal taxane as described above. In therapeutic use for the treatment of cancer, the liposome compositions of the present invention can be administered such that the initial dosage of the taxane ranges from about 0.001 mg/kg to about 1000 mg/kg daily. A daily dose of about 0.01-500 mg/kg, or about 0.1 to about 200 mg/kg, or about 1 to about 100 mg/kg, or about 10 to about 50 mg/kg, or about 10 mg/kg, or about 5 mg/kg, or about 2.5 mg/kg, or about 1 mg/kg can be used. Further, a daily dose of about 3, about 6, about 12, about 24, about 48, about 80, about 120, about 160, about 190, about 225, about 270, about 320 and about 380 mg/m² can be used.

The dosages may be varied depending upon the requirements of the patient, the type and severity of the cancer being treated, and the pharmaceutical composition being employed. For example, dosages can be empirically determined considering the type and stage of cancer diagnosed in a particular patient. The dose administered to a patient should be sufficient to affect a beneficial therapeutic response in the patient over time. The size of the dose will also be determined by the existence, nature and extent of any adverse side-effects that accompany the administration of a particular liposome composition in a particular patient. Determination of the proper dosage for a particular situation is within the skill of the practitioner. Generally, treatment is initiated with smaller dosages which are less than the optimum dose of the liposome composition. Thereafter, the dosage is increased by small increments until the optimum effect under circumstances is reached. For convenience, the total daily dosage may be divided and administered in portions during the day, if desired. The duration of the infusion may be extended and/or the infusion may be interrupted in the case of an adverse event, but the total duration of the infusion cannot exceed 2 hours and cannot be resumed for several hours following the initiation of the infusion.

The methods described herein apply especially to solid tumor cancers (solid tumors), which are cancers of organs and tissue (as opposed to hematological malignancies), and ideally epithelial cancers. Examples of solid tumor cancers include bile duct cancer, bladder cancer, breast cancer, cervical cancer, colorectal cancer (CRC), esophageal cancer, gastric cancer, head and neck cancer, hepatocellular cancer, lung cancer, melanoma, neuroendocrine cancer, ovarian cancer, pancreatic cancer, prostate cancer, renal cancer and thymus cancer. In one group of embodiments, the solid tumor cancer suitable for treatment according to the methods of the invention are selected from CRC, breast cancer and prostate cancer. In another group of embodiments, the methods of the invention apply to treatment of hematological malignancies, including for example multiple myeloma, T-cell lymphoma, B-cell lymphoma, Hodgkins disease, non-Hodgkins lymphoma, acute myeloid leukemia and chronic myelogenous leukemia.

The pharmaceutical compositions may be administered alone in the methods of the invention, or in combination with other therapeutic agents. The additional agents can be anticancer agents belonging to several classes of drugs such as, but not limited to, cytotoxic agents, VEGF-inhibitors, tyrosine kinase inhibitors, monoclonal antibodies and immunotherapies. Examples of such agents include, but are not limited to, doxorubicin, cisplatin, oxaliplatin, carboplatin, 5-fluorouracil, gemcitabine (anti-metabolite), ramucirumab (VEGF 2 inhibitor), bevacizumab, trastuzumab (monoclonal antibody HER2 inhibitor), afatinib (EGFR tyrosine kinase inhibitor) and others. Additional anti-cancer agents can include, but are not limited to, 20-epi-1,25 dihydroxyvitamin D3,4-ipomeanol, 5-ethynyluracil, 9-dihydrotaxol, abiraterone, acivicin, aclarubicin, acodazole hydrochloride, acronine, acylfulvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizing morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisaziridinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caracemide, carbetimer, carboplatin, carboxamide-amino-triazole, carboxyamidotriazole, carest M3, carmustine, cam 700, cartilage derived inhibitor, carubicin hydrochloride, carzelesin, casein kinase inhibitors, castanospermine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethylnorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocarmycin SA, ebselen, ecomustine, edatrexate, edelfosine, edrecolomab, eflomithine, eflomithine hydrochloride, elemene, elsamitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, fluorocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ilmofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-M, interferons, interleukins, iobenguane, iododoxorubicin, iproplatin, irinotecan, irinotecan hydrochloride, iroplact, irsogladine, isobengazole, isohomohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levami sole, liarozole, liarozole hydrochloride, linear polyamine analog, lipophilic disaccharide peptide, lipophilic platinum compounds, lissoclinamide 7, lobaplatin, lombricine, lometrexol, lometrexol sodium, lomustine, lonidamine, losoxantrone, losoxantrone hydrochloride, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C inhibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, 06-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazofurin, pyrazoloacridine, pyri doxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RII retinamide, RNAi, rogletimide, rohitukine, romurtide, roquinimex, rubiginone B1, ruboxyl, safingol, safingol hydrochloride, saintopin, sarcnu, sarcophytol A, sargramostim, SDI 1 mimetics, semustine, senescence derived inhibitor 1, sense oligonucleotides, signal transduction inhibitors, signal transduction modulators, simtrazene, single chain antigen binding protein, sizofuran, sobuzoxane, sodium borocaptate, sodium phenylacetate, solverol, somatomedin binding protein, sonermin, sparfosate sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine, vinorelbine tartrate, vinrosidine sulfate, vinxaltine, vinzolidine sulfate, vitaxin, vorozole, zanoterone, zeniplatin, zilascorb, zinostatin, zinostatin stimalamer or zorubicin hydrochloride.

### Pharmaceutical Compositions

Pharmaceutical compositions of the present invention generally contain liposomal formulations as described herein and a pharmaceutically acceptable carrier. The term “carrier” typically refers to a inert substance used as a diluent or vehicle for the liposomal formulation. The term also encompasses a typically inert substance that imparts cohesive qualities to the composition. Typically, the physiologically acceptable carriers are present in liquid form. Examples of liquid carriers include, but not limited to, physiological saline, phosphate buffer, normal buffered saline (135-150 mM NaCl), water, buffered water, 0.4% saline, 0.3% glycine, 0.3M sucrose (and other carbohydrates), glycoproteins to provide enhanced stability (e.g., albumin, lipoprotein, globulin, etc.) and the like. Since physiologically acceptable carriers are determined in part by the particular composition being administered as well as by the particular method used to administer the composition, there are a wide variety of suitable formulations of pharmaceutical compositions of the present invention (see, e.g., Remington's Pharmaceutical Sciences, Maak Publishing Company, Philadelphia, Pa., 17th ed. (1985)).

The compositions of the present invention may be sterilized by conventional, well-known sterilization techniques or may be produced under sterile conditions. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents, wetting agents and the like, e.g., sodium acetate, sodium lactate, sodium chloride, potassium chloride, calcium chloride, sorbitan monolaurate and triethanolamine oleate. Sugars can also be included for stabilizing the compositions, such as a stabilizer for lyophilized liposome compositions.

Pharmaceutical compositions suitable for parenteral administration, such as, for example, by intraarticular, intravenous, intramuscular, intratumoral, intradermal, intraperitoneal and subcutaneous routes, include aqueous and non-aqueous, isotonic sterile injection solutions. The injection solutions can contain antioxidants, buffers, bacteriostats and solutes that render the formulation isotonic with the blood of the intended recipient, and aqueous and non-aqueous sterile suspensions that can include suspending agents, solubilizers, thickening agents, stabilizers and preservatives. Injection solutions and suspensions can also be prepared from sterile powders, such as lyophilized liposomes. In the practice of the present invention, compositions can be administered, for example, by intravenous infusion, intraperitoneally, intravesically or intrathecally. Parenteral administration and intravenous administration are preferred methods of administration. The formulations of liposome compositions can be presented in unit-dose or multi-dose sealed containers, such as ampoules and vials.

The pharmaceutical composition is preferably in unit dosage form. In such form, the composition is subdivided into unit doses containing appropriate quantities of the active component, e.g., a liposome formulation. The unit dosage form can be a packaged composition, the package containing discrete quantities of the pharmaceutical composition. The composition can, if desired, also contain other compatible therapeutic agents.

### In Vivo Pharmacokinetics Properties of the Liposomal Pharmaceutical Compositions

The liposomal pharmaceutical composition disclosed herein may be formulated for oral, intravenous, intramuscular, intraperitoneal or rectal delivery. Bioavailabilty is often assessed by comparing standard pharmacokinetic (PK) parameters such as Cₘₐₓ and AUC.

In one embodiment, the liposomal pharmaceutical composition may produce a plasma PK profile characterized by docetaxel plasma levels above the putative efficacy threshold for Taxotere® (e.g., 0.2 μM) for about 1 hour to about 125 hours, about 5 hours to about 100 hours, about 5 hour to about 75 hours, about 10 hours to 50 hours or about 20 to about 40 hours. In another embodiment, the C_{inf} may be above the efficacy threshold for about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120 or about 125 hours.

In one embodiment, the liposomal pharmaceutical composition may produce a plasma PK profile characterized by docetaxel plasma levels 2 times above the putative efficacy threshold for Taxotere® (e.g., 0.4 μM) for about 1 hour to about 60 hours, about 2 hours to about 55 hours, about 3 hour to about 50 hours, about 4 hours to 45 hours, about 10 to about 40 hours or about 20 to about 40 hours. In another embodiment, the Cₘₐₓ may be above the efficacy threshold for about 1, about 2, about 3, 4, about 5, about 6, about 7, about 8, about 9, about 10, about 15, about 20, about 25, about 30, about 35, about 40, about 45, about 50, about 55 or about 60 hours.

In one embodiment, the liposomal pharmaceutical composition may produce a plasma PK profile characterized by Cₘₐₓ for docetaxel from about 10 ng/ml to about 5,000 ng/ml, from about 25 ng/ml to about 4,500 ng/ml, from about 50 mg/ml to about 4,000 ng/ml, from about 75 ng/ml to about 3,000 ng/ml, from about 100 ng/ml to about 2,500 ng/ml, from about 150 ng/ml to about 2,000 ng/ml, from about 200 ng/ml to about 1,500 ng/ml, from about 300 ng/ml to about 1,000 ng/ml or from about 300 ng/ml to about 500 ng/ml. In another embodiment, the Cₘₐₓ for docetaxel may be about 10, about 20, about 30, about 40, about 50, about 75, about 100, about 150, about 200, about 250, about 300, about 350, about 400, about 450, about 500, about 600, about 700, about 800, about 900, about 1,000, about 1,500, about 2,000, about 2,500, about 3,000, about 3,500, about 4,000, about 4,500 or about 5,000 ng/ml.

In an additional embodiment, an additional embodiment, the liposomal pharmaceutical composition may produce a plasma PK profile characterized by AUC for docetaxel from about 10,000 ng·hr/ml to about 200,000 ng·hr/ml, from about 10,000 ng·hr/ml to about 175,000 ng·hr/ml, from about 10,000 ng·hr/ml to about 150,000 ng·hr/ml, from about 10,000 ng·hr/ml to about 125,000 ng·hr/ml, from about 10,000 ng·hr/ml to about 100,000 ng·hr/ml, from about 10,000 ng·hr/ml to about 75,000 ng·hr/ml, from about 10,000 ng·hr/ml to about 55,000 ng·hr/ml, from about 15,000 ng·hr/ml to about 45,000 ng·hr/ml, from about 20,000 ng·hr/ml to about 40,000 ng·hr/ml or from about 25,000 ng·hr/ml to about 30,000 ng·hr/ml. In another embodiment, the AUC for docetaxel may be about 10,000, about 15,000, about 20,000, about 25,000, about 30,000, about 35,000, about 40,000, about 45,000, about 50,000, about 55,000, about 60,000, about 65,000, about 70,000, about 75,000, about 80,000, about 85,000, about 90,000, about 95,000, about 100,000, about 125,000, about 150,000, about 175,000 or about 200,000 ng·hr/ml

In an additional embodiment, the liposomal pharmaceutical composition may produce a plasma PK profile characterized by dose normalized (AUC_{inf}_{_}_{D}) for docetaxel from about 100 h*m²*ng/ml/mg to about 500 h*m²*ng/ml/mg, from about 125 h*m²*ng/ml/mg to about 450 h*m²*ng/ml/mg, from about 150 h*m²*ng/ml/mg to about 350 h*m²*ng/ml/mg, from about 200 h*m²*ng/ml/mg to about 300 h*m²*ng/ml/mg, from about 250 h*m²*ng/ml/mg to about 350 h*m²*ng/ml/mg or from about 350 h*m²*ng/ml/mg to about 475 h*m²*ng/ml/mg. In another embodiment, the dose normalized (AUC_{inf}_{_}_{D}) for docetaxel may be about 100, about 125, about 150, about 175, about 200, about 225, about 250, about 275, about 300, about 325, about 350, about 375, about 400, about 425, about 450, about 475 or about 500 h*m²*ng/ml/mg.

In an additional embodiment, the liposomal pharmaceutical composition may produce a plasma PK profile characterized by t_{1/2} for docetaxel from about 15 hours to about 75 hours, from about 15 hours to about 65 hours, from about 15 hours to about 55 hours, from about 20 hours to about 50 hours, from about 25 hours to about 45 hours or from about 25 hours to about 40 hours. In another embodiment, the t_{1/2} for docetaxel from may be about 15, about 20, about 25, about 30, about 35, about 40, about 45, about 50, about 55, about 60, about 65, about 70 or about 75 hours.

In an additional embodiment, the liposomal pharmaceutical composition may produce a plasma PK profile characterized by clearance (CL) for docetaxel below about 30 L/h/m², about 29 L/h/m², about 28 L/h/m², about 27 L/h/m², about 26 L/h/m², about 25 L/h/m², about 24 L/h/m², about 23 L/h/m², about 22 L/h/m², about 21 L/h/m², about 20 L/h/m², about 19 L/h/m², about 18 L/h/m², about 17 L/h/m², about 16 L/h/m², about 15 L/h/m², about 14 L/h/m², about 13 L/h/m², about 12 L/h/m², about 11 L/h/m², about 10 L/h/m², about 9 L/h/m², about 8 L/h/m², about 7 L/h/m², about 6 L/h/m², about 5 L/h/m², about 4 L/h/m², about 3 L/h/m², about 2 or about 1 L/h/m². In still another embodiment, the liposomal composition may produce a plasma PK profile characterized by CL for docetaxel below about 5 L/h/m², about 4.75 L/h/m², about 4.5 L/h/m², about 4.25 L/h/m², about 4 L/h/m², about 3.75 L/h/m², about 3.5 L/h/m², about 3.25 L/h/m², about 3 L/h/m², about 2.75 L/h/m², about 2.5 L/h/m², about 2.25 L/h/m², about 2 L/h/m², about 1.75 L/h/m², about 1.5 L/h/m², about 1.25 L/h/m² or about 1 L/h/m².

In one embodiment, the liposomal pharmaceutical composition may produce a plasma PK profile characterized by Cₘₐₓ for TD-1 from about 1,000 ng/ml to about 500,000 ng/ml, from about 1,000 ng/ml to about 450,000 ng/ml, from about 1,000 ng/ml to about 400,000 ng/ml, from about 5,000 ng/ml to about 350,000 ng/ml, from about 5,000 ng/ml to about 300,000 ng/ml from about 5,000 ng/ml to about 250,000 ng/ml, from about 10,000 mg/ml to about 200,000 ng/ml, from about 15,000 ng/ml to about 150,000 ng/ml, from about 20,000 ng/ml to about 100,000 ng/ml or from about 25,000 ng/ml to about 50,000 ng/ml. In another embodiment, the Cₘₐₓ for TD-1 may be about 1,000, about 10,000, about 15,000, about 20,000, about 25,000, about 30,000, about 35,000, about 40,000, about 45,000, about 50,000, about 55,000, about 60,000, about 65,000, about 70,000, about 75,000, about 80,000, about 85,000, about 90,000, about 95,000, about 100,000, about 110,000, about 120,000, about 130,000, about 140,000, about 150,000, about 160,000, about 170,000, about 180,000, about 190,000, about 200,000, about 225,000, about 250,000, about 275,000, about 300,000, about 325,000, about 350,000, about 375,000, about 400,000, about 425,000, about 450,000, about 475,000 or about 500,000 ng/ml.

In an additional embodiment, an additional embodiment, the liposomal pharmaceutical composition may produce a plasma PK profile characterized by AUC_{inf} for TD-1 from about 100,000 ng·hr/ml to about 45,000,000 ng·hr/ml, from about 150,000 ng·hr/ml to about 40,000,000 ng·hr/ml, from about 200,000 ng·hr/ml to about 35,000,000 ng·hr/ml, from about 250,000 ng·hr/ml to about 30,000,000 ng·hr/ml, from about 300,000 ng·hr/ml to about 25,000,000 ng·hr/ml, from about 400,000 ng·hr/ml to about 20,000,000 ng·hr/ml, 500,000 ng·hr/ml to about 15,000,000 ng·hr/ml, 600,000 ng·hr/ml to about 10,000,000 ng·hr/ml, from about 700,000 ng·hr/ml to about 5,000,000 ng·hr/ml, from about 800,000 ng·hr/ml to about 4,000,000 ng·hr/ml, from about 900,000 ng·hr/ml to about 3,000,000 ng·hr/ml or from about 1,000,000 ng·hr/ml to about 2,000,000 ng·hr/ml. In another embodiment, the AUC for docetaxel may be about 100,000, about 150,000, about 200,000, about 250,000, about 300,000, about 350,000, about 400,000, about 450,000, about 500,000, about 600,000, about 700,000, about 800,000, about 900,000, about 1,000,000, about 2,000,000, about 3,000,000, about 4,000,000, about 5,000,000, about 6,000,000, about 7,000,000, about 8,000,000, about 9,000,000, about 10,000,000, about 11,000,000, about 12,000,000, about 13,000,000, about 14,000,000, about 15,000,000, about 20,000,000, about 25,000,000, about 30,000,000, about 35,000,000, about 40,000,000 or about 45,000,000 ng·hr/ml

In an additional embodiment, the liposomal pharmaceutical composition may produce a plasma PK profile characterized by dose normalized (AUC_{inf}_{_}_{D}) for TD-1 from about 10,000 h*m²*ng/ml/mg to about 1,250,000 h*m²*ng/ml/mg, 10,000 h*m²*ng/ml/mg to about 1,000,000 h*m²*ng/ml/mg, from about 15,000 h*m²*ng/ml/mg to about 900,000 h*m²*ng/ml/mg, from about 20,0000 h*m²*ng/ml/mg to about 800,000 h*m²*ng/ml/mg, from about 25,000 h*m²*ng/ml/mg to about 700,000 h*m²*ng/ml/mg, from about 30,000 h*m²*ng/ml/mg to about 600,000 h*m²*ng/ml/mg, from about 35,000 h*m²*ng/ml/mg to about 500,000 h*m²*ng/ml/mg, from about 40,000 h*m²*ng/ml/mg to about 400,000 h*m²*ng/ml/mg, from about 45,000 h*m²*ng/ml/mg to about 400,000 h*m²*ng/ml/mg, from about 50,000 h*m²*ng/ml/mg to about 300,000 h*m²*ng/ml/mg or from about 100,000 h*m²*ng/ml/mg to about 200,000 h*m²*ng/ml/mg. In another embodiment, the dose normalized (AUC_{inf D}) for docetaxel may be about 10,000, about 20,000, about 30,000, about 40,000, about 50,000, about 60,000, about 70,000, about 80,000, about 90,000, about 100,000, about 150,000, about 200,000, about 250,000, about 300,000, about 350,000, about 400,000, about 450,000, about 500,000, about 550,000, about 600,000, about 750,000, about 800,000, about 850,000, about 900,000, about 950,000, about 1,000,000 or about 1,250,000 h*m²*ng/ml/mg.

In an additional embodiment, the liposomal pharmaceutical composition may produce a plasma PK profile characterized by t_{1/2} for TD-1 from about 15 hours to about 100 hours, from about 15 hours to about 90 hours, from about 15 hours to about 85 hours, from about 15 hours to about 75 hours, from about 15 hours to about 65 hours, from about 15 hours to about 55 hours, from about 20 hours to about 50 hours, from about 25 hours to about 45 hours, from about 25 hours to about 40 hours, from about 35 hours to about 55 hours or from about 45 hours to about 60 hours. In another embodiment, the t_{1/2} for docetaxel from may be about 15, about 20, about 25, about 30, about 35, about 40, about 45, about 50, about 55, about 60, about 65, about 70, about 75, about 80, about 85, about 90, about 95 or about 100 hours.

In an additional embodiment, the liposomal pharmaceutical composition may produce a plasma PK profile characterized by CL for TD-1 below about 0.1 L/h/m², about 0.09 L/h/m², about 0.08 L/h/m², about 0.07 L/h/m², about 0.6 L/h/m², about 0.05 L/h/m², about 0.04 L/h/m², about 0.03 L/h/m², about 0.02 L/h/m² or about 0.01 L/h/m².

### IV. Examples

### Example 1

### Pharmacokinetic and Biodistribution of Liposomal Taxane Derivative in Mice with A549 Xenograft, Comparative Results

Pharmacokinetic and tissue distribution studies have been completed in tumor bearing mice comparing PEGylated TD-1 liposomes with Taxotere® (docetaxel). The PEGylated TD-1 liposomes contain a prodrug of docetaxel (TD-1) to improve solubility, tolerability and increase efficacy through improved pharmacokinetics and biodistribution. Docetaxel is a lipophilic cytotoxin which is not well retained within liposomes. In contrast, TD-1 possesses enhanced hydrophilicity, which prevents the compound from crossing the liposomal lipid bilayer. Without wishing to be bound by any theory, under acidic conditions (pH ˜5), it is believed that the prodrug remains stable and retained within the aqueous interior of the liposome (Zhigaltsev et al, 2010). Once introduced into the circulation, the luminal pH of the liposome slowly increases and the prodrug hydrolyzes to the active metabolite, docetaxel. The increased lipophilicity of docetaxel allows this cytotoxin to easily cross the lipid bilayer of the liposome and then enter the systemic circulation or extracellular space of a tumor. The biodistribution and pharmacokinetics of PEGylated TD-1 liposomes in immunodeficient mice bearing A549 Human Non-Small Cell Lung Carcinoma (NSCLC) xenograft was evaluated to determine whether PEGylated TD-1 liposomes produces greater systemic exposure and tumor accumulation of docetaxel compared to the standard of care Taxotere®.

### Pharmacokinetics

The plasma pharmacokinetics and distribution were studied in female athymic nude mice each implanted subcutaneously with A549 cells (human non-small cell lung cancer). Once tumors reached a volume of 100-300 mm³, animals were randomized into 4 groups. Each animal was given a single intravenous dose of docetaxel or PEGylated TD-1 liposomes as shown in Table 1.

TABLE 1
Dosing Assignments for Nude Mice Bearing A459 Xenograft

Dose (mg/kg)

| Test Article | No. Animals | TD-1 ^{a} | Docetaxel ^{b} |
|---|---|---|---|
| Docetaxel | 27 | 0 | 30 |
| Docetaxel | 27 | 0 | 50 |
| PEGylated TD-1 | 27 | 50 | 40 |
| liposomes | | | |
| PEGylated TD-1 | 27 | 180 | 144 |
| liposomes | | | |

^{a} = Test article dose is expressed as mg/kg TD-1
^{b} = Test article dose is expressed as mg/kg docetaxel equivalent (TD-1/1.25 conversion factor)

Three animals were sacrificed at 1, 4, 24, 72 (3 days), 168 (7 days), 216 (9 days), 336 (14 days), 432 (18 days) and 504 hours (21 days) post injection. Blood samples were taken for pharmacokinetic analysis at each time point (dichlorvos and formic acid were added within 15 minutes of collection to prevent conversion of TD-1 to docetaxel). Pharmacokinetic parameters of TD-1 and docetaxel were calculated using the Phoenix WinNonLin software by non-compartment analysis modeling.

The plasma concentrations of TD-1 and docetaxel decreased over time after intravenous administration of PEGylated TD-1 liposomes, as shown in FIG. 1A and FIG. 1B. At a dose of 40 mg/kg PEGylated TD-1 liposomes, TD-1 concentrations remained above the limits of quantitation (0.025 μg/mL) through 168 hours (7 days) after liposome administration; whereas, following a dose of 144 mg/kg PEGylated TD-1 liposomes, TD-1 was detected through the entire three week observation period after liposome administration (FIG. 1A). The long circulating prodrug resulted in circulating docetaxel levels of four and seven days post dose of 40 and 144 mg/kg PEGylated TD-1 liposomes, respectively, compared to just four hours after administration of 30 or 50 mg/kg docetaxel (FIG. 1B).

The Cₘₐₓ and systemic exposure (plasma AUC) of TD-1 increased with an increase in the dose of PEGylated TD-1 liposomes (Table 2). Further, PEGylated TD-1 liposomes demonstrate short clearance (CL) and a small volume of distribution (Vd).

TABLE 2
Pharmacokinetics of TD-1 Following Administration of PEGylated
TD-1 Liposomes to Nude Mice Bearing A549 Xenograft

| Dose (mg/kg) | 40 | 144 |
|---|---|---|
| t_{1/2} (h) | 10.5 | 10.5 |
| Cₘₐₓ (μg/mL) | 786 | 2907 |
| AUC_{∞} (μg · h/mL) | 20920 | 112682 |
| CL (mL/h/kg) | 2.4 | 1.6 |
| Vd (mL/kg) | 36 | 24 |

PEGylated TD-1 liposomes (40 mg/kg) exhibited Cₘₐₓ docetaxel concentrations similar to those resulting from the administration of docetaxel (50 mg/kg) itself but the exposure, in terms of AUC, was almost 10 times greater (Table 3). PEGylated TD-1 liposomes provided a reservoir for the continual slow sustained release in the circulation and in tumors of docetaxel.

TABLE 3
Pharmacokinetics of Docetaxel Following Administration of PEGylated
TD-1 Liposomes to Nude Mice Bearing A549 Xenograft

| | Compound | | | |
|---|---|---|---|---|
| | Docetaxel | | PEGylated TD-1 liposomes | |
| Dose (mg/kg) | 30 | 50 | 40 | 144 |
| t_{1/2} (h) | * | * | 12 | 16 |
| Cₘₐₓ (μg/mL) | 2.7 | 8.6 | 10 | 36 |
| AUC_{∞} (μg · h/mL) | 8.8 | 27 | 267 | 1146 |
| CL (mL/h/kg) | * | * | 148 | 126 |
| Vd (mL/kg) | 16110 | 37187 | 2531 | 2848 |

* = Not calculable

The docetaxel derived from PEGylated TD-1 liposomes appeared to be restricted to a smaller volume of distribution compared to docetaxel administered as the free drug. The plasma concentration of docetaxel generated from PEGylated TD-1 liposomes was approximately 1% that of TD-1 measured in the blood through 3 days post dose.

Traditional chemotherapeutics, for instance Taxotere®, act by killing cells that divide rapidly (a key property of cancer cells). In short, the strategy is to kill the cancer cells before the patient. In such cases, dosing frequency depends on the patient's recovery time. However, key PK parameters, such as AUC, clearance (CL) and half-life (t_{1/2}), are not optimized but simply ignored. Indeed, the unfavorable PK profile associated with high toxicity (as shown in FIG. 2) has a profound negative impact on the therapeutic index of docetaxel.

As shown in FIG. 2, there is a therapeutic window in which the docetaxel drug level can effectively treat disease while staying within the safety range (i.e., maximum tolerated dose or MTD). Generally, a Cₘₐₓ above 0.64 μg/ml and an AUC greater than 1.42 μg*hr/ml are associated with increased incidence of adverse effects. When administered systemically, Taxotere® produces a sharp and high peak in plasma concentrations of docetaxel which are associated with adverse effects, including neutropenia, hypersensitivity reactions, fluid retention, peripheral neuropathy, myelosuppression, gastrointestinal toxicity, etc.

The PEGylated TD-1 liposomes, however, provide a reservoir for the continual slow sustained release of docetaxel in the circulation and in tumors with levels above the efficacy threshold¹ but below the toxicity threshold. This allows for maximum therapeutic efficacy and safety (i.e., optimal Cₘₐₓ and AUC) of docetaxel over a longer period of time (t_{1/2}). ¹ The putative efficacy threshold was determined as described in Clarke and Rivory, Clin. Pharmacokinet. 36:99-114 (1999), Bruno et al., J. Clin. Oncol. 16:187-196 (1998), and http://www.cancerrxgene.org/translation/Drug/1007.

Tissue Distribution in Mice with A549 Xenograft

In addition to the plasma levels and pharmacokinetic calculations, an assessment of tissue distribution was done in A549 human NSCLC tumor bearing mice after the administration of PEGylated TD-1 liposomes (as in Table 1).

TD-1 accumulated in the A549 tumors for an extended period of time (FIG. 3A). The concentration of TD-1 increased slowly through the first 24 hours after injection. After 24 hours, concentrations of TD-1 tended to drift downward with time at the low dose. At the high dose, concentrations remained somewhat stable through approximately 14 days post dose and then tended to increase but the variability also increased. The concentration of TD-1 remained above the lower limits of quantitation (2.0 μg/g) through the 21 day observation period.

Similarly, administration of PEGylated TD-1 liposomes resulted in increasing concentrations of docetaxel in the A549 tumors through the first 7 days for low dose (40 mg/kg) and through 9 days for the high dose (144 mg/kg). After the initial peak, docetaxel concentrations decreased slightly and then remained stable through the remainder of the 21 day observation period following the low dose (FIG. 3B). After the high dose of PEGylated TD-1 liposomes, concentrations of docetaxel decreased slightly and again increased 18 and 21 days after dosing. For both doses, PEGylated TD-1 liposomes produced sustained TD-1 and docetaxel levels over a 21 day observation period in A549 NSCLC xenograft tumors from athymic nude mice. In contrast, intravenous injection of docetaxel peaked immediately after injection in all tissues. Tumor levels of docetaxel decreased with time falling below the levels of quantitation (1.0 μg/g) after nine days. PEGylated TD-1 liposomes (40 and 144 mg/kg) produced 4 and 18 fold greater docetaxel exposure in tumor, respectively, compared to administration of docetaxel.

At comparable doses, PEGylated TD-1 liposomes (40 mg/kg) exhibited a tumor exposure (AUC) of docetaxel 3.9 times greater than the administration of docetaxel (50 mg/kg) itself (Table 4).

TABLE 4
Levels of Docetaxel in Tissue Following Administration
of Docetaxel or PEGylated TD-1 Liposomes to
Nude Mice Bearing A549 Xenograft

| | Compound | | | |
|---|---|---|---|---|
| | Docetaxel | | PEGylated TD-1 liposomes | |
| Dose (mg/kg) | 30 | 50 | 40 | 144 |
| Tumor AUC (μg · h/g) | 276 | 442 | 1744 | 7955 |
| Liver AUC (μg · h/g) | 10 | 37 | 1320 | 2838 |
| Spleen AUC (μg · h/g) | 77 | 162 | 402 | 3606 |
| Kidney AUC (μg · h/g) | 28 | 179 | 1164 | 2546 |
| Lung AUC (μg · h/g) | 86 | 211 | 21 | 592 |
| Muscle AUC (μg · h/g) | 23 | 64 | BLQ | BLQ |

BLQ = Below Levels of Quantitation

In the tumor, the docetaxel levels following administration of PEGylated TD-1 liposomes (expressed as a percent of the docetaxel level following administration of unencapsulated TD-1) increased after 3 to 7 days, particularly at the lower dose where the level reached 55% after 21 days. The ratio was generally stable in other tissues and ranged from around 1-2% in the liver and spleen up to 3-5% in the kidneys.

Levels of TD-1 in the liver, spleen, kidney, lung and skeletal muscle tissue appeared to fall into two categories (FIG. 4A and FIG. 4B). The liver, spleen and kidney showed a pattern similar to the tumor with a slow uptake through the first 72 hours with concentrations slowly decreasing through the remainder of the 3 week period. The lung and skeletal muscle tissue contained the highest concentrations immediately after injection which decreased to concentrations close to the levels of detection after approximately 72 and 24 hours, respectively.

After approximately nine days, TD-1 concentrations in skeletal muscle tissue fell below the levels of quantitation for the 40 mg/kg dose of PEGylated TD-1 liposomes. A similar pattern of uptake and distribution for TD-1 occurred after the administration of PEGylated TD-1 liposomes at a dose of 144 mg/kg. After the high dose of PEGylated TD-1 liposomes, the lung and skeletal muscle tissue retained measurable concentrations of TD-1 throughout the observation period, but the concentrations tended to be lower than those found for the tumor, liver, spleen and kidney especially through the plateau period between 168 and 504 hours. The limits of quantitation of TD-1 were 0.5 μg/g for the liver, kidney, spleen and lung, and 2.0 μg/g for the skeletal muscle.

The uptake and elimination patterns for docetaxel derived from PEGylated TD-1 liposomes fell into two categories (FIG. 5A and FIG. 5B). PEGylated TD-1 liposomes at doses of 40 or 144 mg/kg failed to produce quantifiable amounts of docetaxel in skeletal muscle tissue. The limits of quantitation for docetaxel were 0.5 μg/g for the liver, kidney, spleen and lung, and 1.0 μg/g for the skeletal muscle. In contrast, docetaxel (50 mg/kg) produced peak tissue docetaxel levels greater than PEGylated TD-1 liposomes at 40 or 144 mg/kg in muscle, lung, spleen, kidney or liver (FIG. 6). However, the concentrations of docetaxel fell below the limits of quantitation after 24 hours for most of the tissues except for the tumor which retained measurable levels of docetaxel through 216 hours (9 days).

Overall, PEGylated TD-1 liposomes (40 mg/kg) produced greater total exposure (AUC) than docetaxel (50 mg/kg) in all tissue except lung and muscle. PEGylated TD-1 liposomes at 144 mg/kg produced greater exposure in all tissue except muscle compared to docetaxel (50 mg/kg).

### Example 2

### In Vivo Anti-Tumor Activity, Comparative Results

Antitumor activity of PEGylated TD-1 liposomes on the growth of established human PC3 xenograft in male immunodeficient mice was studied to determined whether PEGylated TD-1 liposomes could provide greater efficacy than Taxotere® (docetaxel) at equivalent maximum tolerated doses (MTD).

Tumor cell lines were implanted subcutaneously into the flank of nude (immunodeficient) mice and allowed to grow to a fixed size. Mice that did not grow tumors were rejected. Mice were allocated to receive either saline (control, included in all studies) or docetaxel or PEGylated TD-1 liposomes, and administered the designated treatment by slow bolus intravenous injection. In each case, where possible, doses were selected as providing equivalent levels of toxicity/tolerance. The highest doses of TD-1 were usually limited by the volume that could be administered. Tumor volume was analyzed to determine tumor growth delay (TGD) and partial regression. Mice were removed from the study if they lost 20% of their initial bodyweight or became moribund or if their tumor volume exceeded 2500 mm³ or the tumor ulcerated. If less than half of the initial cohort of mice remained, that group was no longer graphed or included in further tumor analysis. However, any remaining animals were followed until completion of the in-life observation period and included in a survival analysis. The variable features of this study are summarized in Table 5.

TABLE 5
Summary of Variable Features of In Vivo Antitumor
Activity Studies in Immunodeficient Mice

| | | | Doses (mg docetaxel/kg) | | |
|---|---|---|---|---|---|
| | Cell | | | TD-1 | PEGylated TD-1 |
| Tumor | Line | No./group | Docetaxel | liposome | liposome |
| Prostate | PC3 | 6 | 9, 18, 27^{a} | 30, 58, 88 | 19, 38, 57 |

^{a}= Prostate PC3 docetaxel (27 mg/kg) dose group had five mice.

The study demonstrate that PEGylated TD-1 liposomes act as an active antitumor agent in this xenograft model, and possess significantly greater antitumor activity compared to comparably tolerated doses of docetaxel.

Data from the study with PC3 prostate tumor model demonstrate that PEGylated TD-1 liposomes possess antitumor activity greater than docetaxel when given at equitoxic doses. A single dose of PEGylated TD-1 liposomes (19, 38, or 57 mg/kg) caused a significant (p<0.05) reduction in tumor volume compared to saline treated mice. While 18 and 27 mg/kg docetaxel also inhibited tumor growth, PEGylated TD-1 liposomes exhibited greater antitumor effects as determined by TGD and partial tumor regression (Table 6). PEGylated TD-1 liposomes significantly (p<0.05) increased survival at each dose evaluated, and 57 mg/kg PEGylated TD-1 liposomes increased survival significantly (p<0.05) when compared to all doses of docetaxel. Notably, the PEGylated TD-1 liposomes exhibited greater tumor volume inhibition than the non-PEGylated TD-1 liposomes. Treatment with PEGylated TD-1 liposomes at 19 mg/kg caused significantly smaller tumors than the equitoxic dose of docetaxel (9 mg/kg) and TD-1 liposomes (30 mg/kg), *p<0.05. Effects on tumor growth and survival are illustrated in FIG. 7A and FIG. 7B.

TABLE 6
Efficacy and Survival Parameters in Mice Bearing
PC3 Xenograft Tumors Following Treatment with Docetaxel,
TD-1 Liposomes or PEGylated TD-1 Liposomes

| | | | | Partial Tumor | Median |
|---|---|---|---|---|---|
| | | TGD | TGI | Regression | Survival |
| Treatment and Dose | TGD | (%) | (%) | (%) | (Days) |
| Saline | — | — | — | 0 | 35 |
| Docetaxel (9 mg/kg) | 11 | 42 | 38 | 0 | 47 |
| Docetaxel (18 mg/kg) | 41 | 154 | 91 | 33 | 81 |
| Docetaxel (27 mg/kg) | 42 | 157 | 98 | 60 | 84 |
| TD-1 liposomes (30 | 21 | 78 | 53 | 17 | 57 |
| mg/kg) | | | | | |
| TD-1 liposomes (58 | 59 | 221 | 99 | 0 | 77 |
| mg/kg) | | | | | |
| TD-1 liposomes (88 | 62 | 233 | 101 | 50 | 104 |
| mg/kg) | | | | | |
| PEGylated TD-1 | —^{a} | —^{a} | 80 | 17 | 56 |
| liposomes (19 mg/kg) | | | | | |
| PEGylated TD-1 | 66 | 250 | 100 | 67 | 89 |
| liposomes (38 mg/kg) | | | | | |
| PEGylated TD-1 | 71 | 268 | 101 | 83 | 126 |
| liposomes (57 mg/kg) | | | | | |

^{a}Tumors treated with 24 mg/kg PEGylated TD-1 liposomes did not reach a target size of 1 cm³, and were excluded from TGD and % TGD.

In another study, athymic male nude mice bearing PC3 human prostate xenograft were given two or four intravenous (IV) doses of PEGylated TD-1 liposome, Taxotere® or saline. Dosing intervals were twenty-one days for two cycles or every four days for four cycles. The doses of Taxotere® and PEGylated TD-1 liposomes were based on maximum tolerated dose (MTD) or highest dose tested for a given dose interval. A summary of the dose groups is provided in Table 7.

TABLE 7
Summary of Dose Groups

| | | | Dose | Docetaxel | Cum. |
|---|---|---|---|---|---|
| | Dose | No. | Volume | Eq. | Dose |
| Test Article | Schedule | Animals | (ml/kg) | (mg/kg) | (mg · kg) |
| Saline | 1, 5, 9, 13 | 10 | 23 | 0 | 0 |
| Docetaxel | 1, 5, 9, 13 | 10 | 5 | 5 | 20 |
| Docetaxel | 1, 5, 9, 13 | 10 | 10 | 10 | 40 |
| Docetaxel | 1, 21 | 10 | 15 | 30 | 60 |
| Docetaxel | 1, 21 | 10 | 30 | 60 | 120 |
| PEGylated TD-1 | 1, 5, 9, 13 | 10 | 6 | 30 | 120 |
| liposomes | | | | | |
| PEGylated TD-1 | 1, 5, 9, 13 | 10 | 12 | 60 | 240 |
| liposomes^{a} | | | | | |
| PEGylated TD-1 | 1, 21 | 10 | 12, 24^{b} | 60 | 120 |
| liposomes | | | | | |
| PEGylated TD-1 | 1, 21 | 10 | 23 | 120 | 240 |
| liposomes | | | | | |

^{a}PEGylated TD-1 liposomes 60 mg/kg, q4d x4 was not tolerated and not included in tumor analysis.
^{b}A different lot of PEGylated TD-1 liposomes was administered to this group on day 21.

Tumor volume was measured 2-3 times per week using the Biopticon tumor imaging system and tumor volume data was analyzed to determine TGD and partial tumor regression. Survival analysis was conducted and median survival time determined. The results are provided in Table 8.

TABLE 8
Efficacy and Survival Parameters in Mice Bearing
PC3 Xenograft Tumors Following Treatment with
Docetaxel, or PEGylated TD-1 Liposomes

| | | | Partial Tumor | Peak Mean |
|---|---|---|---|---|
| | | TGD | Regression | Body Weight |
| Treatment and Dose | TGD | (%) | (%) | Loss |
| Saline | — | — | 0 | −8% |
| Docetaxel 5 mg/kg (q4dx4) | 34 | 213 | 0 | −22% |
| Docetaxel 10 mg/kg(q4dx4) | 69 | 431 | 80 | −23% |
| Docetaxel 30 mg/kg | 73 | 456 | 100 | −19% |
| (q21dx2) | | | | |
| Docetaxel 60 mg/kg | 86 | 538 | 100 | −22% |
| (q21dx2) | | | | |
| PEGylated TD-1 liposomes | 145 | 906 | 90 | −12% |
| 30 mg/kg (q4dx4) | | | | |
| PEGylated TD-1 liposomes | 103 | 644 | 100 | −9% |
| 60 mg/kg (q21dx2) | | | | |
| PEGylated TD-1 liposomes | — | — | 100 | −24% |
| 120 mg/kg (q21dx2) | | | | |

As seen in Table 8, all dose groups of PEGylated TD-1 liposomes partially regressed tumors and delayed growth of tumors to 1000 mm³ by 103 to 145 days compared to saline control as seen by TGD. PEGylated TD-1 liposomes increased TGD 20% and 69% greater than the docetaxel dose group (60 mg/kg) with the greatest TGD. Tumors in mice treated with PEGylated TD-1 liposomes 120 mg/kg (q21dx2) did not reach a target size of 1000 mm³ and were excluded from TGD and % TGD. PEGylated TD-1 liposomes dose groups of 30 and 60 mg/kg decreased mouse body weights similarly to saline treated mice (9% and 12% vs. 8%). 120 mg/kg PEGylated TD-1 liposomes decreased body weight similar to docetaxel at 60 mg/kg (24% vs. 22%).

PEGylated TD-1 liposomes and docetaxel dose dependently inhibited growth of PC3 human prostate xenograft in athymic nude mice as shown by mean tumor volume (mm³) over time after IV administration of docetaxel, PEGylated TD-1 liposomes or saline (FIG. 8A). All dose groups of PEGylated TD-1 liposomes inhibited tumor growth longer than all dose groups of docetaxel. PEGylated TD-1 liposomes doses are given as docetaxel molar equivalents. Further, all dose groups of PEGylated TD-1 liposomes (157, 125, 177 days) increased median survival of mice greater than docetaxel (62, 88, 93, 107 days) and saline (26 days) treatment as seen in Kaplan-Meier Plot showing percent survival of athymic nude mice bearing human PC3 (prostate) xenograft tumors (FIG. 8B). Lastly, all treatment groups transiently decreased body weight after each dose and then recovered to baseline once dose administration was complete (FIG. 8C).

PEGylated TD-1 liposomes produced better efficacy than docetaxel at equitoxic doses in a PC3 human prostate xenograft mouse model. Indeed, all dose groups of PEGylated TD-1 liposomes produced partial tumor regression and delayed growth of tumors longer than docetaxel by 20 to 69%, which resulted in greater survival rates compared to docetaxel.

### Example 3

### Dose Escalation Human Study

A two-part open-label, dose escalation first-in-human (FIH) study in subjects with recurrent and/or metastatic advanced solid malignancies refractory to conventional therapy was initiated to evaluate the safety and tolerability profile, assess the Dose-Limiting Toxicity (DLT), and establish the maximum-tolerated dose (MTD) of PEGylated TD-1 liposomes. A secondary objective was to characterize the pharmacokinetic profile (PK) of docetaxel and the liposomal components (DSPE-PEG[2000]) and TD-1, as well as the preliminary antitumor activity of PEGylated TD-1 liposomes.

PEGylated TD-1 liposomes were administered intravenously (IV) every 21 days for four cycles.² Thirteen dose levels were studied: 3, 6, 12, 24, 48, 80, 120, 160, 190, 240, 270, 320 and 380 mg/m². In part A, the safety, tolerability, MTD, DLTs, PK profile and preliminary antitumor activity of ascending doses of PEGylated TD-1 liposomes was evaluated using a modified “3+3” dose escalation design in an effort to determine the recommended phase II dose, i.e., the dose level immediately below MTD. In part B, at the expansion phase, the recommended phase II dose will be administered to an additional 20 subjects with recurrent and/or metastatic Squamous Cell Carcinoma of the Head and Neck (SCCHN) to further evaluate the safety, PK profile, and preliminary antitumor activity of the PEGylated TD-1 liposomes in the SCCHN population. ² Subjects who have a tumor response after 4 cycles or are deemed to receive clinical benefit from treatment with PEGylated TD-1 liposomes will be allowed to continue to receive PEGylated TD-1 liposomes as part of a long-term extension study.

To date, forty-four subjects have received at least one dose of the PEGylated TD-1 liposome. Preliminary efficacy results are set forth in Table 9. They include eight stable diseases in different tumor types including thymic cancer, Non-Small Cell Lung Cancer (NSCLC), prostate, ovarian, cervical, gastroesophageal cancer, cancer of unknown primary origin and cholangiocarcinoma.

TABLE 9
Summary of Efficacy Results

| | | | Initial Dose | End Dose | | |
|---|---|---|---|---|---|---|
| Age | Gender | Cancer Type | (mg/m²) | (mg/m²) | Response | No. Doses |
| 63 | M | Thymus* | 3 | 120 | SD | 22 |
| 70 | M | NSCLC | 6 | 6 | SD | 6 |
| 69 | M | Prostate | 12 | 12 | SD | 7 |
| 48 | F | Ovarian* | 12 | 12 | SD | 6 |
| 60 | F | Cervical* | 24 | 24 | SD | 5 |
| 58 | M | Gastroesophageal | 80 | 80 | SD | 6 |
| 51 | F | Unknown Primary* | 160 | 160 | SD | 5 |
| 56 | F | NSCLC | 190 | 190 | SD | 4 |
| 74 | F | Ovarian* | 320 | 320 | PR | 6 |
| 53 | M | Cholangiocarcinoma* | 320 | 320 | SD | 4 |

*Not currently indicated for Taxotere ®

As shown in Table 9, nine patients had their disease stabilized after 4 cycles of treatment with MNK-010. Stable disease (SD) is defined as neither sufficient shrinkage to qualify for partial response nor sufficient increase to qualify for progressive disease. Two patients had a partial response (PR) with MNK-010. A partial response is defined as a 30% decrease in the sum of the diameters of target lesions. One PR was confirmed at the end of 4 cycles (i.e. was observed on two consecutive radiologic evaluations at least 6 weeks apart), but the second partial response remained unconfirmed (at the end of 2 cycles and one radiologic evaluation) as the patient was still active in the study. The confirmed partial response was observed in an ovarian cancer patient and the unconfirmed partial response was observed in a patient with head and neck cancer of unknown primary origin.

The plasma concentration of docetaxel at various dose level is shown in FIG. 9A-FIG. 9C. The PK profile for TD-1 after one cycle is provided in Table 10 below. FIG. 10A and FIG. 10B show the correlation between the peak docetaxel concentration (Cₘₐₓ) and exposure (AUC_{0-inf}) versus dose (mg/m²)

TABLE 10
Summary of PK Parameters for Docetaxel

| | Dose (mg/m²) | | | | | | | |
|---|---|---|---|---|---|---|---|---|
| Parameter | 3 | 6 | 12 | 24 | 48 | 80 | 120 | 160 |
| Cₘₐₓ (ng/ml) | 15.5 | 36.3 | 74.7 | 149.0 | 187.3 | 307.0 | 701.7 | 878.8 |
| Cₘₐₓ/Dose | 5.17 | 6.06 | 6.22 | 6.21 | 3.90 | 3.84 | 5.85 | 5.49 |
| (m² · ng/ml/mg) | | | | | | | | |
| AUC_{0-inf} | 225 | 265 | 721 | 4899 | 5297 | 4798 | 28103 | 16518 |
| (ng · h/ml) | | | | | | | | |
| AUC/Dose | 75.1 | 44.1 | 60.1 | 204.1 | 110.4 | 60.0 | 234.2 | 103.2 |
| (m² · ng · h/ml/mg) | | | | | | | | |
| CL (l/h/m²) | 23.73 | 24.65 | 16.92 | 8.75 | 13.29 | 21.61 | 7.15 | 10.46 |
| Vss (l/m²) | 261.8 | 272.4 | 257.1 | 389.8 | 705.3 | 736.2 | 292.5 | 573.7 |
| t_{1/2} (h) | 13.99 | 9.32 | 12.26 | 55.28 | 73.53 | 28.77 | 45.84 | 51.11 |

| | Dose (mg/m²) | | | | |
|---|---|---|---|---|---|
| Parameter | 190 | 225 | 270 | 320 | 380 |
| Cₘₐₓ (ng/ml) | 1126 | 1044 | 1190 | 1737 | 2900 |
| Cₘₐₓ/Dose | 5.93 | 4.64 | 4.41 | 5.43 | 7.63 |
| (m² · ng/ml/mg) | | | | | |
| AUC_{0-inf} | 18774 | 33923 | 26153 | 37627 | 106700 |
| (ng · h/ml) | | | | | |
| AUC/Dose | 98.8 | 150.8 | 96.9 | 117.6 | 280.8 |
| (m² · ng · h/ml/mg) | | | | | |
| CL (l/h/m²) | 10.33 | 6.75 | 10.35 | 9.14 | 3.56 |
| Vss (l/m²) | 549.2 | 413.4 | 451.7 | 404.0 | 327.7 |
| t_{1/2} (h) | 51.93 | 61.75 | 35.50 | 40.80 | 67.72 |

The maximum plasma docetaxel concentrations (Cₘₐₓ) ranged, on average, from 1190 ng/mL to 2900 ng/mL on Cycle 1, Day 1 in patients administered 270 mg/m² to 380 mg/m² PEGylated TD-1 liposomes. Further, Cₘₐₓ was similar to and half-life was longer (2900 ng/mL; 380 mg/m²; t_{1/2}—51 h overall) than that seen following high dose Taxotere® (2680 ng/mL; 100 mg/m²; 10-19 h) (see, e.g., van Oosterom, AT; Schriivers, D. Docetaxel (Taxotere®), a Review of Preclinical and Clinical Experience. Part 2: Clinical Experience. Anti-Cancer Drugs 1995, 6, 356-368).

The plasma concentration of TD-1 at various dose level is shown in FIG. 11A and FIG. 11B. The PK profile for docetaxel after one cycle is provided in Table 11 below. FIG. 12A and FIG. 12B show the correlation between the peak TD-1 concentration (Cₘₐₓ) versus dose (mg/m²) and exposure (AUC_{0-inf}) versus dose (mg/m²).

TABLE 10
Summary of PK Parameters for TD-1

| | Dose (mg/m²) | | | | | | |
|---|---|---|---|---|---|---|---|
| Parameter | 3 | 6 | 12 | 24 | 48 | 80 | 120 |
| Cₘₐₓ (ng/ml) | 1520 | 3150 | 6475 | 13700 | 30733 | 43367 | 68833 |
| Cₘₐₓ/Dose | 506.7 | 525.0 | 539.6 | 570.8 | 640.3 | 542.1 | 573.6 |
| (m² · ng/ml/mg) | | | | | | | |
| AUC_{0-inf} | 102546 | 252313 | 457271 | 954790 | 2662532 | 2387921 | 6279141 |
| (ng · h/ml) | | | | | | | |
| AUC/Dose | 34182 | 42052 | 38106 | 39783 | 55469 | 29849 | 52326 |
| (m² · ng · h/ml/mg) | | | | | | | |
| CL (l/h/m²) | 0.0367 | 0.0274 | 0.0283 | 0.0260 | 0.0186 | 0.0343 | 0.0202 |
| Vss (l/m²) | 2.17 | 1.67 | 1.46 | 1.45 | 1.40 | 1.24 | 0.090 |
| t_{1/2} (h) | 48.75 | 57.58 | 44.01 | 43.77 | 59.27 | 26.70 | 48.26 |

| | Dose (mg/m²) | | | | | |
|---|---|---|---|---|---|---|
| Parameter | 160 | 190 | 225 | 270 | 320 | 380 |
| Cₘₐₓ (ng/ml) | 98450 | 143000 | 149000 | 150333 | 205000 | 310000 |
| Cₘₐₓ/Dose | 615.3 | 752.6 | 662.2 | 556.8 | 640.6 | 815.8 |
| (m² · ng/ml/mg) | | | | | | |
| AUC_{0-inf} | 9440039 | 9808251 | 13178503 | 8371853 | 12975255 | 34121587 |
| (ng · h/ml) | | | | | | |
| AUC/Dose | 59000 | 51622 | 58571 | 31007 | 405478 | 897934 |
| (m² · ng · h/ml/mg) | | | | | | |
| CL (l/h/m²) | 0.0182 | 0.0195 | 0.0184 | 0.0330 | 0.0260 | 0.011 |
| Vss (l/m²) | 1.37 | 1.22 | 1.08 | 1.59 | 1.58 | 1.25 |
| t_{1/2} (h) | 66.09 | 52.96 | 54.52 | 35.61 | 43.74 | 84.36 |

The average t_{1/2} for the TD-1 was approximately 51 h. Both docetaxel and TD-1 Cₘₐₓ and AUC remained linear with respect to dose.

FIG. 13A, FIG. 13B and FIG. 14 illustrate the plasma concentration of docetaxel relative to the putative efficacy threshold at different dose levels of PEGylated TD-1 liposomes.

Since the PEGylated TD-1 liposomes cannot be measured directly, TD-1 and the lipid component DSPE(PEG-2000) were measured as surrogates for PEGylated TD-1 liposomes. The mean plasma concentrations are shown in FIG. 15A, FIG. 15B, FIG. 16A and FIG. 16B. Specifically, FIG. 15A and FIG. 16A illustrate the mean plasma concentrations for TD-1, and FIG. 15B and FIG. 16B illustrate the mean plasma concentrations for DSPE(PEG-2000). The docetaxel, DSPE(PEG-2000) and TD-1 demonstrate dose proportionality for Cₘₐₓ and AUC_{inf} (FIG. 17A, FIG. 17B, FIG. 18A, FIG. 18B, FIG. 19A, and FIG. 19B, respectively). Since Cₘₐₓ and AUC demonstrate dose proportionality for TD-1, DSPE(PEG-2000), and docetaxel, PEGylated TD-1 liposomes, in turn, demonstrate good dose proportionality.

The clearance (CL), volume of distribution (Vₛₛ), half-life (t_{1/2}), peak level (Cₘₐₓ), and extent of exposure (AUC) values were comparable between TD-1 and DSPE(PEG-2000) for dose levels 3 to 380 mg/m². The mean pharmacokinetic parameters for TD-1 and DSPE(PEG-2000) are provided in Table 11 below. The CL (0.025 and 0.025 L/h/m²) and Vₛₛ (1.422 vs 1.409 L/m²) for TD-1 and DSPE-PEG(2000) are very similar, indicating that the prodrug is mostly associated with the liposomes and has an identical disposition as DSPE-PEG(2000), and that PEGylated TD-1 liposomes have a very short clearance and small tissue distribution. The mean t_{1/2} of both species is about 50 hours. Cₘₐₓ and AUC demonstrate dose proportionality for TD-1, DSPE-PEG(2000) and docetaxel. The dose normalized Cₘₐₓ of docetaxel released from PEGylated TD-1 liposome is several fold lower and the AUC is about two fold greater relative to the Cₘₐₓ and AUC reported for Taxotere® (docetaxel) (see Clarke & Rivory. Clin Pharmacokinet. 1999, 36: 99-114; Taxotere® Prescribing Information, Sanofi-Aventis, May 2014; both incorporated by reference herein). The t_{1/2} of released docetaxel is over 3 fold longer (42 hours vs 12 hours) than reported t_{1/2} for Taxotere® (docetaxel).

TABLE 11
Mean (SD) PK Parameters of TD-1,
DSPE(PEG-2000) and Docetaxel

| | | | | |
|---|---|---|---|---|
| t_{1/2} | Cmax-D | AUCinf-D | CL | Vss |
| (hour) | (m²*ng/ml/mg) | (h*m²*ng/ml/mg) | (L/h/m²) | (L/m²) |

TD-1

| | | | | | |
|---|---|---|---|---|---|
| Mean | 50.04 | 601.51 | 46162 | 0.025 | 1.422 |
| SD | 18.43 | 104.42 | 17442 | 0.010 | 0.415 |

DSPE(PEG-2000)

| | | | | | |
|---|---|---|---|---|---|
| Mean | 52.54 | 633.68 | 47791 | 0.025 | 1.409 |
| SD | 29.89 | 181.95 | 24755 | 0.010 | 0.468 |

Docetaxel

| | | | | | |
|---|---|---|---|---|---|
| Mean | 41.72 | 5.29 | 118.03 | 14.13 | 458.8 |
| SD | 29.37 | 1.40 | 92.29 | 9.58 | 250.8 |

Safety data shows that the PEGylated TD-1 liposome is well tolerated at doses up to 380 mg/m² with only three cases of thrombocytopenia and three cases of neutropenia to date. FIG. 20 illustrates the dose versus neutrophil counts in subjects treated with PEGylated TD-1 liposomes. Further, as shown in FIG. 20-FIG. 24B, no correlation between dose or Cₘₐₓ or AUC_{inf} to neutrophil and platelets was observed, and no severe hemotologic toxicity was evident.

No new side effects of PEGylated TD-1 liposome were expected to be contributed by docetaxel, the active moiety of this product. Adverse Events (AE) were evaluated and categorized in accordance with the National Cancer Institute (NCI) Common Terminology Criteria for Adverse Events (CTCAE, version 4.03 [2010]). Table 12 provides a summary of the most frequent adverse events for Grade 1 (mild) and Grade 2 (Moderate).

TABLE 12
Most Frequent Adverse Events (Grade 1 and 2)

| | | Number of |
|---|---|---|
| Adverse Events | Causality | Events |
| Fatigue | Possible | 17 |
| Peripheral Neuropathy | Related | 9 |
| Nausea | Possible | 8 |
| Infusion-related reactions | Possible | 7 |
| Vomiting | Possible | 6 |
| Rash | Related | 5 |
| Leukopenia | Possible | 5 |
| Generalized Weakness | Possible | 3 |
| Thrombocytopenia | Possible | 3 |
| AST/ALT Elevation | Possible | 3 |
| Neutropenia | Possible | 3 |

The major drug-related Grade 1 and 2 adverse events reported were fatigue, peripheral neuropathy, nausea, infusion-related reactions and vomiting.

Table 13 provides a summary of the most frequent adverse events Grade 3 or 4.

TABLE 13
Most Frequent Adverse Events (Grade 3 and 4)

| Adverse Events | Grade | Causality |
|---|---|---|
| Fatigue | 3 | Possible |
| Neuropathy | 3 | Possible |
| Diarrhea | 3 | Possible |
| Worsening Fatigue | 3 | Possible |
| Lymphopenia | 3 | Possible |
| Diarrhea - DLT | 3 | Possible |
| Peripheral Neuropathy | 3 | Possible |
| Anemia | 3 | Possible |
| Elevated Transaminase | 3 | Possible |
| Abdominal Pain | 3 | Possible |
| Toxic Epidermal Necrolysis | 3 | Possible |

The major drug-related Grade 3 adverse events reported were fatigue, neuropathy/peripheral neuropathy and others. One case of Grade 3 peripheral neuropathy was reported in one subject after administration of 22 cycles. The event resolved to a Grade 2 within 21 days. A total of eleven Grade 3 but no Grade 4 or higher toxicities were reported. Diarrhea and abdominal pain accompanied by elevated liver transaminase are the dose-limiting toxicities in this study.

The PEGylated TD-1 liposomes act as a drug depot with the slow conversion and release of docetaxel resulting in a relatively lower Cₘₐₓ and enhanced systemic exposure (AUC) over a prolonged period of time. This unique PK profile will improve efficacy as well as a better safety profile when compared to docetaxel.

### Example 4

### Liposomal Formulation

The following PEGylated TD-1 liposomal formulations were prepared by the methods of the present invention.

TABLE 14
PEGylated TD-1 Liposomal Formulations

| | Particle | | Total | | | % | | Free TD-1 | % | | | |
|---|---|---|---|---|---|---|---|---|---|---|---|---|
| | size | | lipids | % | % | DSPE- | TD-1 | (includes | Free | Docetaxel | % | TD1/ |
| Description | (nm) | pH | (mg/mL) | PC | Chol | PEG2000 | mg/mL | Docetaxel) | TD-1 | mg/mL | Docetaxel | Lipids |
| DSPC/DSPE/Chol | 115.5 | 6.78 | 19.9 | 51.8% | 45.9% | 2.4% | 3.7 | 0.01 | 0.3% | 0.036 | 1.0% | 0.19 |
| (45/10/45) | | | | | | | | | | | | |
| with DSPE-PEG(2000) | | | | | | | | | | | | |
| DOPC/Chol (55/45) | 108.2 | 6.70 | 20.7 | 51.3% | 44.2% | 4.5% | 3.79 | 0.09 | 2.4% | 0.066 | 1.7% | 0.18 |
| with DSPE-PEG(2000) | | | | | | | | | | | | |
| POPC/Chol (75/25) | 89.26 | 6.79 | 18.3 | 75.3% | 19.4% | 5.4% | 3.33 | 3.96 | 119.0% | 0.077 | 2.3% | 0.18 |
| with DSPE-PEG(2000) | | | | | | | | | | | | |
| DOPC/Chol (65/35) | 86.50 | 6.80 | 23.4 | 62.4% | 32.6% | 5.0% | 4.11 | 0.35 | 8.5% | 0.097 | 2.4% | 0.18 |
| with DSPE-PEG(2000) | | | | | | | | | | | | |
| HSPC/Chol (55/45) | 104.5 | 6.79 | 17.9 | 60.5% | 35.8% | 3.8% | 3.11 | 0.01 | 0.3% | 0.034 | 1.1% | 0.17 |
| with DSPE-PEG(2000) | | | | | | | | | | | | |
| DSPC/Chol (55/45) | 109.0 | 6.81 | 20.2 | 56.0% | 40.6% | 3.4% | 3.47 | 0.01 | 0.3% | 0.052 | 1.5% | 0.17 |
| with DSPE-PEG(2000) | | | | | | | | | | | | |
| DMPC/Chol (55/45) | 91.39 | 6.78 | 20.7 | 55.6% | 41.9% | 2.5% | 3.43 | 0.18 | 5.2% | 0.044 | 1.3% | 0.17 |
| with DSPE-PEG(2000) | | | | | | | | | | | | |
| DSPC/Chol (55/45) | 114.7 | 6.81 | 17.0 | 62.1% | 34.5% | 3.5% | 2.8 | 0.01 | 0.3% | 0.037 | 1.3% | 0.16 |
| with DSPE-PEG(5000) | | | | | | | | | | | | |
| DSPC/Chol (65/35) | 91.71 | 6.79 | 16.8 | 74.4% | 23.7% | 1.9% | 2.7 | 0.01 | 0.5% | 0.043 | 1.6% | 0.16 |
| with DSPE-PEG(2000) | | | | | | | | | | | | |
| DPPC/Chol (55/45) | 106.5 | 6.83 | 20.7 | 60.0% | 37.0% | 3.1% | 3.2 | 0.03 | 0.9% | 0.041 | 1.3% | 0.15 |
| with DSPE-PEG(2000) | | | | | | | | | | | | |
| SOPC/Chol (55/45) | 99.8 | 6.75 | 19.1 | 52.6% | 42.8% | 4.6% | 2.89 | 0.63 | 21.8% | 0.040 | 1.4% | 0.15 |
| with DSPE-PEG(2000) | | | | | | | | | | | | |
| SOPC/Chol (55/45) | 102.9 | 6.85 | 18.1 | 52.4% | 42.7% | 4.9% | 2.72 | 1.03 | 37.9% | 0.023 | 0.8% | 0.15 |
| with DSPE-PEG(2000) | | | | | | | | | | | | |
| POPC/Chol (55/45) | 96.63 | 6.90 | 22.1 | 60.0% | 36.1% | 4.0% | 3.08 | 0.77 | 25.0% | 0.030 | 1.0% | 0.14 |
| with DSPE-PEG(2000) | | | | | | | | | | | | |
| HSPC/Chol (65/35) | 100.1 | 6.85 | 15.7 | 70.4% | 26.5% | 3.1% | 2.18 | 0.02 | 1.0% | 0.042 | 1.9% | 0.14 |
| with DSPE-PEG(2000) | | | | | | | | | | | | |
| POPC/Chol (65/35) | 101.4 | 6.84 | 22.3 | 73.5% | 21.4% | 5.2% | 2.71 | 2.52 | 93.0% | 0.111 | 4.1% | 0.12 |
| with DSPE-PEG(2000) | | | | | | | | | | | | |
| DSPC/DSPG/Chol | 177.2 | 6.74 | 14.2 | 51.1% | 48.9% | 0.0% | 1.24 | 0.01 | 0.8% | 0.049 | 4.0% | 0.09 |
| (45/10/45) | | | | | | | | | | | | |
| with DSPE-PEG(2000) | | | | | | | | | | | | |
| SOPC/Chol (65/35) | 91.83 | 6.82 | 20.4 | 62.9% | 32.0% | 5.2% | 1.59 | 1.25 | 78.6% | 0.057 | 3.6% | 0.08 |
| with DSPE-PEG(2000) | | | | | | | | | | | | |
| DSPC/Chol (75/25) | 98.08 | 6.79 | 17.6 | 80.7% | 18.2% | 1.1% | 0.51 | 0.02 | 3.8% | 0.019 | 3.7% | 0.03 |
| with DSPE-PEG(2000) | | | | | | | | | | | | |
| DPPC/Chol (65/35) | 96.25 | 6.75 | 14.0 | 78.0% | 22.0% | 0.0% | 0.192 | 0.01 | 3.1% | 0.007 | 3.9% | 0.01 |
| with DSPE-PEG(2000) | | | | | | | | | | | | |
| DMPC/Chol (65/35) | 84.49 | 6.80 | 15.9 | 67.1% | 32.4% | 0.5% | 0.1 | 0.08 | 80.0% | 0.001 | 1.0% | 0.01 |
| with DSPE-PEG(2000) | | | | | | | | | | | | |
| DPPC/Chol (75/25) | 158.5 | 6.85 | 16.2 | 82.9% | 17.1% | 0.0% | 0.1 | 0.02 | 24.3% | 0.010 | 9.7% | 0.01 |
| with DSPE-PEG(2000) | | | | | | | | | | | | |

The liposomal formulations were evaluated for the following properties:
1) encapsulation of TD-1, as measured by the ratio of drug to total lipids. Higher values are indicative of higher levels of remote loading into the vesicles (values less than 0.1 indicate either less than optimal remote loading or loss of drug during the DSPE-PEG insertion step);
2) % of TD-1 that had been released from the formulation (% free), with higher values of % free suggestive of poor retention of drug (>25%);
3) % of docetaxel, with low values indicating successful preparation without significant hydrolysis of the prodrug (>5%);
4) Particle size of the vesicles as an indication of vesicle integrity during processing (particle sizes greater than 120 nm suggestive of extensive changes during processing); and
5) incorporation of DSPE-PEG into the vesicles post-remote loading of TD-1 (low values <1 mole % indicative of poor incorporation).

With the knowledge of the preferred PK profiles for the TD-1 and docetaxel, a liposomal formulation under the present invention can be developed using other combinations of phosphatidylcholine, sterol, PEG-lipid and TD-1 to provide a sustained release of docetaxel.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

What is claimed is:

1. A pharmaceutical composition for the treatment of cancer comprising a liposomal formulation, wherein the liposomal formulation comprises:
i) about 50 mol % to about 70 mol % of a phosphatidylcholine lipid or a mixture of phosphatidylcholine lipid;
ii) about 25 mol % to about 45 mol % of a sterol;
iii) about 2 mol % to about 8 mol % of a PEG-lipid; and
iv) a taxane or a pharmaceutically acceptable salt thereof; wherein the taxane is docetaxel esterified at the 2′-O-position with a heterocyclyl-(C₂₋₅ alkanoic acid); and
v) a pharmaceutically acceptable carrier; and
wherein upon administration of the pharmaceutical composition to a subject in need thereof, the plasma concentration of docetaxel remains above an efficacy threshold of 0.2 μM for at least 5 hours.

2. The pharmaceutical composition of claim 1, wherein the phosphatidylcholine lipid is selected from the group consisting of: 1,2-dioleoyl-sn-glycero-3-phosphocholine (DOPC), 1,2-distearoyl-sn-glycero-3-phosphocholine (DSPC), hydrogenated soy PC (HSPC), 1,2-dipalmitoyl-sn-glycero-3-phosphocholine (DPPC), 1-palmitoyl-2-oleoyl-sn-glycero-3-phosphocholine (palmitoyloleoylphosphatidylcholine (POPC) and 1-palmitoyl-2-linoleoyl-sn-glycero-3-phosphocholine, i-stearoyl-2-oleoyl-sn-glycero-3-phosphocholine (SOPC).

3. The pharmaceutical composition of claim 1, wherein the pharmaceutical composition of claim 1, wherein the phosphatidylcholine lipid is DSPC.

4. The pharmaceutical composition of claim 1, wherein the sterol is cholesterol.

5. The pharmaceutical composition of claim 1, wherein the PEG-lipid is selected from the group consisting of: distearoyl-phosphatidylethanolamine-N-[methoxy(polyethene glycol)-2000] (DSPE-PEG-2000) and distearoyl-phosphatidylethanolamine-N-[methoxy(polyethene glycol)-5000] (DSPE-PEG-5000).

6. The pharmaceutical composition of claim 5, wherein the PEG-lipid is DSPE-PEG-2000.

7. The pharmaceutical composition of claim 1, wherein the liposomal formulation comprises:
i) about 53 mol % of DSPC, about 44 mol % of cholesterol, and about 3 mol % of DSPE-PEG-2000; or
ii) about 66 mol % of DSPC, about 30 mol % of cholesterol, and about 4 mol % of DSPE-PEG-2000.

8. The pharmaceutical composition of claim 1, wherein the plasma concentration of docetaxel remains above an efficacy threshold of 0.2 μM for at least about 6, about 7, about 8, about 9, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, or about 125 hours.

9. The pharmaceutical composition of claim 1, wherein the plasma concentration of docetaxel remains above an efficacy threshold of 0.4 μM for at least 5 hours.

10. The pharmaceutical composition of claim 9, wherein the plasma concentration of docetaxel remains above an efficacy threshold of 0.4 μM for at least about 6, about 7, about 8, about 9, about 10, about 15, about 20, about 25, about 30, about 35, about 40, about 45, about 50, about 55, or about 60 hours.

11. The pharmaceutical composition of claim 1, wherein upon administration of the pharmaceutical composition to a subject in need thereof, the pharmaceutical composition produces a plasma PK profile characterized by any of the following:
i) AUC_{inf} for docetaxel from about 10,000 ng·hr/ml to about 100,000 ng·hr/ml;
ii) AUC_{inf}_{_}_{D} for docetaxel from about 100 h*m²*ng/ml/mg to about 500 h*m²*ng/ml/mg;
iii) t_{1/2} for docetaxel from about 15 hours to about 75 hours; and/or
iv) CL for docetaxel below about 30 L/h/m².

12. The pharmaceutical composition of claim 1 further comprises a targeting agent or diagnostic agent.

13. A method of treating a cancer comprising administering to a patient in need thereof a pharmaceutical composition comprising a liposomal formulation, wherein the liposomal formulation comprises:
i) about 50 mol % to about 70 mol % of a phosphatidylcholine lipid or a mixture of phosphatidylcholine lipid;
ii) about 25 mol % to about 45 mol % of a sterol;
iii) about 2 mol % to about 8 mol % of a PEG-lipid; and
iv) a taxane or a pharmaceutically acceptable salt thereof; wherein the taxane is docetaxel esterified at the 2′-O-position with a heterocyclyl-(C₂₋₅ alkanoic acid); and
v) a pharmaceutically acceptable carrier; and
wherein upon administration of the pharmaceutical composition to a subject in need thereof, the plasma concentration of docetaxel remains above an efficacy threshold of 0.2 μM for at least 5 hours.

14. The method of claim 13, wherein the liposomal formulation comprises:
i) about 53 mol % of DSPC, about 44 mol % of cholesterol, and about 3 mol % of DSPE-PEG-2000; or
ii) about 66 mol % of DSPC, about 30 mol % of cholesterol, and about 4 mol % of DSPE-PEG-2000

15. The method of claim 13, wherein the daily dose of the liposomal formulation is from 0.001 mg/kg to about 1000 mg/kg daily.

16. The method of claim 13, wherein the daily dose of the liposomal formulation is about 3, about 6, about 12, about 24, about 48, about 80, about 120, about 160, about 190, about 225, about 270, about 320 and about 380 mg/m².

17. The method of claim 13, wherein the cancer is a solid tumor cancer selected from the group consisting of: bile duct cancer, bladder cancer, breast cancer, cervical cancer, colorectal cancer (CRC), esophageal cancer, gastric cancer, head and neck cancer, hepatocellular cancer, lung cancer, melanoma, neuroendocrine cancer, ovarian cancer, pancreatic cancer, prostate cancer, renal cancer, and thymus cancer.

18. The method of claim 17, wherein the solid tumor cancer is selected from the group consisting of: cervical cancer, CRC, bile duct cancer, breast cancer, lung cancer, ovarian, prostate cancer and thymus cancer.

19. The method of claim 13, wherein the cancer is a hematological malignancies selected from the group consisting of: multiple myeloma, T-cell lymphoma, B-cell lymphoma, Hodgkins disease, non-Hodgkins lymphoma, acute myeloid leukemia, and chronic myelogenous leukemia.

20. The method of claim 13, wherein the liposomal formulation exhibits a tumor exposure (AUC) of docetaxel about 4.0 times greater than the administration of docetaxel.

21. The method of claim 13, wherein the liposomal formulation produced sustained docetaxel levels above 1.0 g/g in vivo over a 21 day period.
